(19) United States
(12) Patent Application Publication
(10) Pub. No.: US 2025/0235151 A1
(43) Pub. Date: Jul. 24, 2025

(54) PHYSICAL CONDITION ABNORMALITY DETERMINATION DEVICE AND PHYSICAL CONDITION ABNORMALITY DETERMINATION METHOD

(71) Applicant: Mitsubishi Electric Corporation, Tokyo (JP)

(72) Inventors: Natsuki TAHARA, Tokyo (JP); Shunya OSAWA, Tokyo (JP)

(73) Assignee: Mitsubishi Electric Corporation, Tokyo (JP)

(21) Appl. No.: 18/703,510

(22) PCT Filed: Dec. 24, 2021
(86) PCT No.: PCT/JP2021/048069

§ 371 Date: Apr. 22, 2024

(51) Int. Cl. A61B 5/00; A61B 5/0205; A61B 5/024; A61B 5/11; G06T 7/00; G06T 7/73

(52) U.S. Cl. CPC A61B 5/4561; A61B 5/0205; A61B 5/1114; A61B 5/4094; A61B 5/7207; G06T 7/0012; G06T 7/73; A61B 5/024; A61B 2503/22; G06T 2207/30201; G06T 2207/30268

(57) ABSTRACT

Included are: a posture collapse detecting unit that detects posture collapse of an occupant in a mobile object on the basis of a head position of the occupant in an imaged image obtained by imaging at least a face of the occupant, the head position being detected on the basis of the imaged image; a skeleton point detecting unit that detects a skeleton coordinate point indicating a body part of the occupant on the imaged image on the basis of the imaged image; a convulsion detecting unit that detects a convulsion of the occupant on the basis of skeleton coordinate point information regarding the skeleton coordinate point detected by the skeleton point detecting unit; and a determination unit that determines that the occupant has a physical condition abnormality when posture collapse of the occupant is not detected and convulsion of the occupant is detected.

### TECHNICAL FIELD

The present disclosure relates to a physical condition abnormality determination device and a physical condition abnormality determination method.

### BACKGROUND ART

An occupant in a mobile object may suddenly have a seizure due to a cerebrovascular disease, a heart disease, epilepsy, or the like, and may have a physical condition abnormality. When the occupant has a physical condition abnormality, the physical condition abnormality may appear in body movement. For example, posture collapse in which a posture of an occupant is largely inclined can be considered as one of body movements that occur when the occupant has a physical condition abnormality. Conventionally, a technique of determining that an occupant has a physical condition abnormality by focusing on this point is known.

For example, Patent Literature 1 discloses a device for detecting a driving incapability state of a driver of a vehicle, which detects posture collapse of the driver on the basis of, for example, inclination of a head of the driver, and determines that the driver is in a driving incapability state when the posture collapse is detected.

### CITATION LIST

### Patent Literature

Patent Literature 1: JP 2016-9257 A

### SUMMARY OF INVENTION

### Technical Problem

Some of physical condition abnormalities of an occupant do not involve a large change in posture that can be said to be posture collapse. In the conventional technique as disclosed in Patent Literature 1, since this is not taken into consideration, there is a problem that it may be impossible to determine that an occupant has a physical condition abnormality when the occupant has a physical condition abnormality not involving a large change in posture that can be said to be posture collapse.

The present disclosure has been made in order to solve the above problem, and an object of the present disclosure is to provide a physical condition abnormality determination device capable of determining a physical condition abnormality of an occupant not involving a large change in posture.

### Solution to Problem

A physical condition abnormality determination device according to the present disclosure includes: a posture collapse detecting unit that detects posture collapse of an occupant in a mobile object on the basis of a head position of the occupant in an imaged image obtained by imaging at least a face of the occupant, the head position being detected on the basis of the imaged image; a skeleton point detecting unit that detects a skeleton coordinate point indicating a body part of the occupant on the imaged image on the basis of the imaged image; a convulsion detecting unit that detects a convulsion of the occupant on the basis of skeleton coordinate point information regarding the skeleton coordinate point detected by the skeleton point detecting unit; and a determination unit that determines that the occupant has a physical condition abnormality when the posture collapse of the occupant is not detected and the convulsion of the occupant is detected.

### Advantageous Effects of Invention

According to the present disclosure, it is possible to determine a physical condition abnormality of an occupant not involving a large change in posture.

### BRIEF DESCRIPTION OF DRAWINGS

FIG. 1 is a diagram illustrating a configuration example of a physical condition abnormality determination device according to a first embodiment.

FIGS. 2A, 2B, and 2C are diagrams for explaining an example of a difference between a part change amount of a person and periodicity of the part change amount when the person does not have a physical condition abnormality, and a part change amount of the person and periodicity of the part change amount when the person has a convulsion.

FIG. 3 is a flowchart for explaining an example of an operation of a calibration process performed by the physical condition abnormality determination device according to the first embodiment.

FIG. 4 is a flowchart for explaining an example of an operation of determining a physical condition abnormality of a driver, performed by the physical condition abnormality determination device according to the first embodiment.

FIG. 5 is a flowchart for explaining a detailed operation of a posture collapse detecting process performed by a posture collapse detecting unit in step ST3 of FIG. 4.

FIG. 6 is a flowchart for explaining a detailed operation of a convulsion detecting process performed by a convulsion detecting unit in step ST5 of FIG. 4.

FIGS. 7A and 7B are diagrams illustrating an example of a hardware configuration of the physical condition abnormality determination device according to the first embodiment.

FIG. 8 is a diagram illustrating a configuration example of a physical condition abnormality determination device according to a second embodiment.

FIG. 9 is a flowchart for explaining an example of an operation of determining a physical condition abnormality of a driver, performed by the physical condition abnormality determination device according to the second embodiment.

FIG. 10 is a flowchart for explaining a detailed operation of a pulse abnormality detecting process performed by a pulse abnormality detecting unit in step ST18 of FIG. 9.

FIG. 11 is a diagram illustrating a configuration example of a physical condition abnormality determination device according to a third embodiment.

FIG. 12 is a flowchart for explaining an example of an operation of determining a physical condition abnormality of a driver, performed by the physical condition abnormality determination device according to the third embodiment.

FIG. 13 is a diagram illustrating a configuration example of a physical condition abnormality determination device according to a fourth embodiment.

FIG. 14 is a flowchart for explaining an example of an operation of determining a physical condition abnormality of a driver, performed by the physical condition abnormality determination device according to the fourth embodiment.

### DESCRIPTION OF EMBODIMENTS

Hereinafter, embodiments of the present disclosure will be described in detail with reference to the drawings.

### First Embodiment

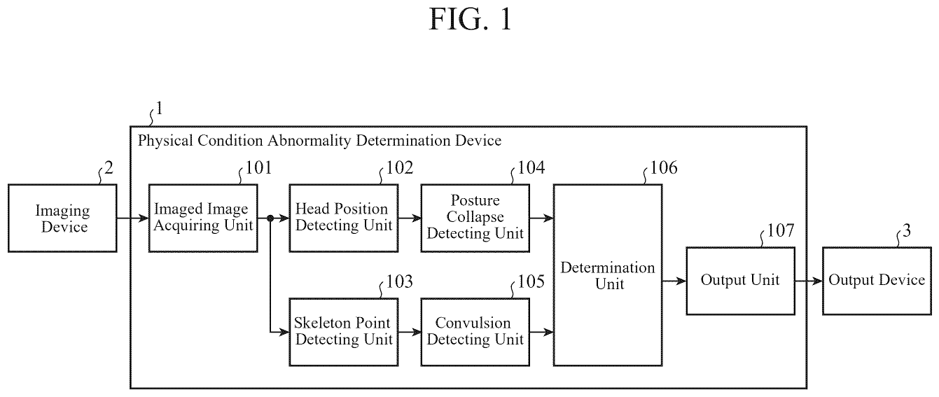

FIG. 1 is a diagram illustrating a configuration example of a physical condition abnormality determination device 1 according to a first embodiment.

The physical condition abnormality determination device 1 according to the first embodiment is assumed to be mounted on a vehicle (not illustrated). The physical condition abnormality determination device 1 is connected to an imaging device 2 and an output device 3.

The imaging device 2 is mounted on the vehicle. The imaging device 2 is disposed in a central portion of an instrument panel of the vehicle, a meter panel thereof, or the like for the purpose of monitoring a vehicle interior. The imaging device 2 is disposed in such a way as to be able to image at least a face of an occupant. In the first embodiment, the imaging device 2 is assumed to be shared with a so-called passenger monitoring system (PMS).

The imaging device 2 is a visible light camera or an infrared camera. In a case where the imaging device 2 is an infrared camera, the infrared camera includes a light source (not illustrated) that emits infrared rays for imaging to a range including a face of an occupant. The light source is constituted by, for example, a light emitting diode (LED).

Note that only one imaging device 2 is disposed in the vehicle in FIG. 1, but this is merely an example. A plurality of imaging devices 2 may be arranged in the vehicle.

The imaging device 2 outputs an image obtained by imaging (hereinafter, referred to as an “imaged image”) to the physical condition abnormality determination device 1.

The physical condition abnormality determination device 1 determines whether or not the occupant in the vehicle has a physical condition abnormality on the basis of the imaged image imaged by the imaging device 2. In the first embodiment, the occupant in the vehicle is assumed to be a driver of the vehicle. When determining that the driver has a physical condition abnormality, the physical condition abnormality determination device 1 outputs information indicating that the driver has a physical condition abnormality (hereinafter, referred to as “physical condition abnormality determining information”) to the output device 3.

In the first embodiment, the physical condition abnormality refers to a sudden change in physical condition that cannot be predicted in advance. Specific examples of the physical condition abnormality include a seizure such as a cerebrovascular disease, a heart disease, or epilepsy. When the driver has a physical condition abnormality, the driver cannot drive. That is, the physical condition abnormality of the driver hinders normal driving of the vehicle, and is likely to cause an unexpected situation. The physical condition abnormality determination device 1 notifies an occupant in the vehicle including the driver, a person around the vehicle, or the like that the driver has a physical condition abnormality by determining a physical condition abnormality of the driver and outputting the physical condition abnormality determining information, which can assist in preventing an unexpected situation.

The output device 3 is, for example, a speaker (not illustrated) mounted on the vehicle. The output device 3 may be, for example, a server (not illustrated) included in a rescue system (not illustrated) outside the physical condition abnormality determination device 1. In addition, for example, when the vehicle is an automatic driving vehicle, the output device 3 may be an automatic driving control device (not illustrated).

When the output device 3 is a speaker, for example, the physical condition abnormality determination device 1 cause the output device 3 to output a warning sound by outputting the physical condition abnormality determining information to the output device 3. As a result, the physical condition abnormality determination device 1 notifies an occupant in the vehicle interior, a person around the vehicle, or the like that the driver has a physical condition abnormality. When the output device 3 is a server, for example, the physical condition abnormality determination device 1 makes a rescue request by outputting the physical condition abnormality determining information to the output device 3. When the output device 3 is an automatic driving control device, for example, the physical condition abnormality determination device 1 can cause the automatic driving control device to perform driving support such as stopping the vehicle or pulling over the vehicle to a road shoulder by outputting the physical condition abnormality determining information to the output device 3.

The physical condition abnormality determination device 1 includes an imaged image acquiring unit 101, a head position detecting unit 102, a skeleton point detecting unit 103, posture collapse detecting unit 104, a convulsion detecting unit 105, a determination unit 106, and an output unit 107.

The imaged image acquiring unit 101 acquires an imaged image from the imaging device 2.

The imaged image acquiring unit 101 outputs the acquired imaged image to the head position detecting unit 102 and the skeleton point detecting unit 103.

In addition, the imaged image acquiring unit 101 stores the acquired imaged image in a storage unit (not illustrated) in association with the acquisition date and time of the imaged image. The storage unit may be included in the physical condition abnormality determination device 1, or may be included in a place that can be referred to by the physical condition abnormality determination device 1 outside the physical condition abnormality determination device 1.

On the basis of the imaged image acquired by imaged image acquiring unit 101, the head position detecting unit 102 detects a head position of the driver in the imaged image.

In the first embodiment, the head position specifically refers to a center of a face area on the imaged image. The face area is an area including all parts of the face such as an outline, an eyebrow, an eye, a nose, and a mouth on the imaged image. In the first embodiment, the face area is, for example, a minimum rectangular area surrounding a contour of the face on the imaged image.

For example, the head position detecting unit 102 detects the face area of the driver using a face detecting unit based on a known general algorithm in which Adaboost or Casecade is combined with a Haar-Like detecting unit. The face detecting unit has learned a large amount of face image data in advance. In addition, for example, the head position detecting unit 102 may detect the face area of the driver using a general method such as so-called model fitting or Elastic Bunch Graph Matching. The head position detecting unit 102 can detect the face area of the driver using various known face recognition techniques on the basis of the imaged image.

When detecting the face area of the driver, the head position detecting unit 102 sets a center of the face area as the head position of the driver. Note that, in the first embodiment, the head position and the face area are represented by coordinates on the imaged image. The face area is represented by, for example, coordinates of four corners of a rectangle of the face area.

The head position detecting unit 102 outputs information regarding the detected head position of the driver (hereinafter, referred to as “head position information”) to the posture collapse detecting unit 104. The head position information includes coordinate information of the head position of the driver in the imaged image. The head position information may include coordinate information of the face area in the imaged image.

The head position information may be, for example, information in which the coordinates of the head position and the coordinates of the face area of the driver on the imaged image are associated with each other, or may be an imaged image to which information capable of specifying the head position of the driver is imparted (hereinafter, referred to as a “head position-imparted imaged image”).

Note that, here, the physical condition abnormality determination device 1 includes the head position detecting unit 102, but this is merely an example. The physical condition abnormality determination device 1 does not necessarily include the head position detecting unit 102. The head position detecting unit 102 may be included in a place that can be referred to by the physical condition abnormality determination device 1 outside the physical condition abnormality determination device 1.

For example, the imaging device 2 may include the head position detecting unit 102. In this case, the imaging device 2 outputs the head position-imparted imaged image to the physical condition abnormality determination device 1. The imaged image acquiring unit 101 acquires the head position-imparted imaged image output from the imaging device 2, and outputs the acquired head position-imparted imaged image to the posture collapse detecting unit 104 and the skeleton point detecting unit 103.

On the basis of the imaged image acquired by imaged image acquiring unit 101, the skeleton point detecting unit 103 detects a skeleton coordinate point indicating a body part of the driver on the imaged image. More specifically, the skeleton point detecting unit 103 detects a skeleton coordinate point of the driver indicating a skeleton point determined for each body part of the driver on the basis of the imaged image acquired by the imaged image acquiring unit 101.

Specifically, the skeleton point detecting unit 103 detects coordinates of the skeleton coordinate point of the driver and a body part of the driver indicated by the skeleton coordinate point. The skeleton coordinate point is a point in the imaged image, and is represented by coordinates in the imaged image.

A body part indicated by a skeleton coordinate point to be detected by the skeleton point detecting unit 103 is determined in advance. The skeleton point detecting unit 103 detects a skeleton coordinate point indicating, for example, both hands, both elbows, both shoulders, a neck, a head, a waist, or both knees. Note that, in the first embodiment, the skeleton point detecting unit 103 is assumed to detect a plurality of skeleton coordinate points of the driver.

The skeleton point detecting unit 103 only needs to detect a skeleton coordinate point of the driver using a known technique. For example, the skeleton point detecting unit 103 detects a skeleton coordinate point of the driver by obtaining information regarding the skeleton coordinate point of the driver using a learned model in machine learning (hereinafter, referred to as a “machine learning model”).

The machine learning model is a machine learning model that receives, as an input, an imaged image obtained by imaging an occupant in the vehicle and outputs information indicating a skeleton coordinate point in the imaged image. The information indicating a skeleton coordinate point includes coordinates of a skeleton coordinate point in the imaged image and information capable of specifying which body part the skeleton coordinate point indicates.

The machine learning model is constructed in such a way as to estimate a result with respect to an input by so-called supervised learning according to learning data generated in advance on the basis of a combination of an input and data of a teacher label. Here, the machine learning model performs learning in such a way as to output information regarding a skeleton coordinate point with respect to an imaged image according to learning data in which the input is an imaged image and the teacher label is information regarding the skeleton coordinate point. Note that the machine learning model performs learning in such a way as to output information regarding a plurality of skeleton coordinate points in an imaged image.

The machine learning model is stored in advance in a place that can be referred to by the skeleton point detecting unit 103.

The skeleton point detecting unit 103 outputs information regarding the detected skeleton coordinate point (hereinafter, referred to as “skeleton coordinate point information”) to the convulsion detecting unit 105. The skeleton coordinate point information includes coordinate information of a skeleton coordinate point in an imaged image and information capable of specifying which body part the skeleton coordinate point indicates.

The skeleton coordinate point information may be, for example, information in which information capable of specifying a skeleton coordinate point is associated with coordinates of each skeleton coordinate point of the driver on the imaged image, or may be an imaged image to which information capable of specifying each skeleton coordinate point of the driver is imparted (hereinafter, referred to as a “skeleton point-imparted imaged image”).

The posture collapse detecting unit 104 detects posture collapse of the driver on the basis of the head position information output from the head position detecting unit 102. Note that, in the first embodiment, the “posture collapse” is assumed to be a large change in posture such as bending forward, turning downward, bending backward, warping like a shrimp, only neck falling sideways, falling sideways, or leaning sideways. In the first embodiment, a process of detecting posture collapse of the driver, performed by the posture collapse detecting unit 104 is referred to as a “posture collapse detecting process”.

In the posture collapse detecting process, the posture collapse detecting unit 104 first detects a change amount of the head position of the driver (hereinafter, referred to as a “posture change amount”). Specifically, the posture collapse detecting unit 104 detects a difference between the head position of the driver specified from the head position information and a reference value (hereinafter, referred to as a “head reference value”) as the posture change amount of the driver. The head reference value is a value (specifically, coordinates on the imaged image) of a head position assumed to be a head position of the driver when the driver does not have a physical condition abnormality, in other words, when the driver is in a normal state.

The head reference value is set in a calibration process performed by the physical condition abnormality determination device 1 prior to the posture collapse detecting process performed by the posture collapse detecting unit 104.

In the calibration process, the physical condition abnormality determination device 1 calibrates the head position of the driver and sets the head reference value. The head reference value set in the calibration process is stored in a place that can be referred to by the posture collapse detecting unit 104. Details of the calibration process will be described later.

The posture collapse detecting unit 104 detects the posture change amount of the driver every time the head position information is output from the head position detecting unit 102. The posture collapse detecting unit 104 stores the detected posture change amount in the storage unit in association with the detection date and time every time the posture collapse detecting unit 104 detects the posture change amount of the driver.

Then, the posture collapse detecting unit 104 determines whether or not a state in which the detected posture change amount of the driver is equal to or more than a preset threshold (hereinafter, referred to as a “posture determining threshold”) continues for a preset time (hereinafter, referred to as “posture determining time”). Note that the posture determining threshold and the posture determining time are set in advance by an administrator or the like, and are stored in a place that can be referred to by the posture collapse detecting unit 104. The posture determining threshold and the posture determining time can be appropriately set. The posture determining time is, for example, ten seconds.

The posture collapse detecting unit 104 detects posture collapse of the driver when a state in which the posture change amount of the driver is equal to or more than the posture determining threshold continues for the posture determining time. That is, the posture collapse detecting unit 104 determines that the driver has posture collapse.

The posture collapse detecting unit 104 does not detect posture collapse of the driver when a state in which the posture change amount of the driver is equal to or more than the posture determining threshold does not continue for the posture determining time. That is, the posture collapse detecting unit 104 determines that the driver does not have posture collapse.

When detecting posture collapse of the driver, the posture collapse detecting unit 104 outputs information indicating that posture collapse of the driver is detected (hereinafter, referred to as “posture collapse occurrence information”) to the determination unit 106. For example, the posture collapse occurrence information may include information regarding the posture change amount of the driver.

When not detecting posture collapse of the driver, the posture collapse detecting unit 104 outputs information indicating that posture collapse of the driver is not detected (hereinafter, referred to as “posture collapse non-occurrence information”) to the determination unit 106.

The convulsion detecting unit 105 detects a convulsion of the driver on the basis of the skeleton coordinate point information output from the skeleton point detecting unit 103. In the first embodiment, a process of detecting a convulsion of the driver, performed by the convulsion detecting unit 105 is referred to as a “convulsion detecting process”.

In general, the “convulsion” refers to a seizure in which a muscle contracts rapidly and involuntarily. The convulsion occurs throughout the body or in some muscles. There are several types of convulsions, and the convulsions are roughly divided into three types of convulsions: a clonic convulsion, a tonic convulsion, and a tonic-clonic convulsion. The clonic convulsion refers to a convulsion that moves a hand, a neck, a head, a foot, or the like in a rattling manner in such a way as to repeat bending and stretching, the tonic convulsion refers to a convulsion that stiffens a limb, and the tonic-clonic convulsion refers to a convulsion combining the clonic convulsion and the tonic convulsion. In the first embodiment, the convulsion detected by the convulsion detecting unit 105 is assumed to be a clonic convulsion or a tonic-clonic convulsion. It is said that a person often has a convulsion when the person has an epileptic seizure. That is, when a person has a convulsion, the person is highly likely to have a physical condition abnormality, and the convulsion can also be said to be a physical condition abnormality.

When a person has a convulsion, the position of a body part moves. However, the movement of the position of a body part due to a convulsion is not as large as movement of the position of a body part in a case of posture collapse. That is, in general, when a person has a convulsion, a large change in posture that can be said to be posture collapse is not observed.

The convulsion detecting unit 105 detects a convulsion not involving a large change in posture that can be said to be posture collapse but can be said to be a physical condition abnormality as in posture collapse.

In the convulsion detecting process, the convulsion detecting unit 105 first detects a change of amount a skeleton coordinate point of the driver (hereinafter, referred to as a “part change amount”). Specifically, the convulsion detecting unit 105 detects a difference between a skeleton coordinate point of the driver specified from the skeleton coordinate point information and a reference value (hereinafter, referred to as a “part reference value”) as the part change amount of the driver. The part reference value is a value (specifically, coordinates on the imaged image) of a skeleton coordinate point assumed to be a skeleton coordinate point of the driver when the driver does not have a physical condition abnormality, in other words, when the driver is in a normal state. Note that the convulsion detecting unit 105 detects a part change amount which is a difference from a corresponding part reference value for each skeleton coordinate point.

The part reference value is set in the calibration process performed by the physical condition abnormality determination device 1 prior to the convulsion detecting process performed by the convulsion detecting unit 105. Note that the physical condition abnormality determination device 1 sets the above-described head reference value and also sets the part reference value in the calibration process.

In the calibration process, the physical condition abnormality determination device 1 calibrates a skeleton coordinate point of the driver and sets the part reference value. At this time, the physical condition abnormality determination device 1 sets the part reference value for each skeleton coordinate point. The part reference value set in the calibration process is stored in a place that can be referred to by the convulsion detecting unit 105 in association with information capable of specifying to which skeleton coordinate point the part reference value corresponds. Details of the calibration process will be described later.

The convulsion detecting unit 105 detects the part change amount of the driver every time the skeleton coordinate point information of the driver is output from the skeleton point detecting unit 103. The convulsion detecting unit 105 stores the detected part change amount in the storage unit in association with the detection date and time every time the convulsion detecting unit 105 detects the part change amount of the driver.

Then, the convulsion detecting unit 105 determines whether or not the detected part change amount of the driver is equal to or more than a preset threshold (hereinafter, referred to as a “convulsion determining threshold”) and periodicity of the part change amount of the driver is periodicity of a convulsion. Note that the convulsion determining threshold and information regarding the periodicity of the part change amount regarded as the periodicity of the convulsion are set in advance by an administrator or the like and stored in a place that can be referred to by the convulsion detecting unit 105. Note that the convulsion detecting unit 105 can determine the periodicity of the part change amount of the driver from the part change amount of the driver stored in the storage unit. The storage unit stores the part change amount in time series. The length of a past period for which the part change amount is used for the convulsion detecting unit 105 to determine the periodicity of the part change amount of the driver is determined in advance.

The convulsion detecting unit 105 detects a convulsion of the driver when the part change amount of the driver is equal to or more than the convulsion determining threshold and the periodicity of the part change amount of the driver in time series is the periodicity of the convulsion. That is, the convulsion detecting unit 105 determines that the driver has a convulsion.

Note that the convulsion detecting unit 105 detects a convulsion of the driver, for example, when there is at least one skeleton coordinate point at which the part change amount is equal to or more than the convulsion determining threshold and the periodicity of the part change amount in time series is the periodicity of the convulsion among the plurality of skeleton coordinate points of the driver.

The convulsion detecting unit 105 does not detect a convulsion of the driver when the part change amount is less than the convulsion determining threshold or when the periodicity of the part change amount in time series is not the periodicity of the convulsion for all the skeleton coordinate points of the driver. That is, the convulsion detecting unit 105 determines that the driver does not have a convulsion.

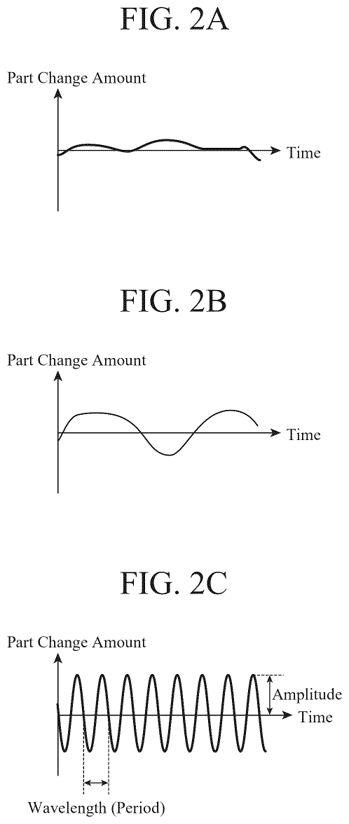

Here, FIGS. 2A, 2B, and 2C are diagrams for explaining an example of a difference between a part change amount of a person and periodicity of the part change amount when the person does not have a physical condition abnormality, in other words, when the person is in a normal state, and a part change amount of the person and periodicity of the part change amount when the person has a convulsion.

FIGS. 2A and 2B are diagrams illustrating a part change amount of a person and periodicity of the part change amount when the person is in a normal state, and FIG. 2C is a diagram illustrating a part change amount of a person and periodicity of the part change amount when the person has a convulsion.

In FIGS. 2A, 2B, and 2C, the vertical axis represents a part change amount, and the horizontal axis represents time.

As illustrated in FIGS. 2A and 2B, when a person is in a normal state, a part change amount (in other words, an amplitude) does not increase so much, and the part change amount does not have periodicity. Meanwhile, when a person has a convulsion, as illustrated in FIG. 2C, a part change amount (in other words, an amplitude) increases and is periodic. A wavelength (period) at this time is short.

As described above, when a person has a convulsion, a part change amount of the person is not such an extent that posture largely changes, but is large to some extent, and the part change amount is periodic. On the basis of this fact, the convulsion detecting unit 105 detects a convulsion of the driver when the part change amount of the driver is equal to or more than the convulsion determining threshold and the periodicity of the part change amount of the driver is the periodicity of the convulsion.

Note that a value based on a part change amount assumed when a person has a convulsion as illustrated in FIG. 2C is set as the convulsion determining threshold. In addition, as information regarding the periodicity of the convulsion, information regarding the periodicity of a part change amount assumed when a person has a convulsion as illustrated in FIG. 2C is set.

Note that, when the convulsion detecting unit 105 detects the convulsion of the driver, the periodicity of the part change amount of the driver and the periodicity of the convulsion do not necessarily coincide with each other completely. The convulsion detecting unit 105 may consider that the periodicity of the part change amount of the driver and the periodicity of the convulsion coincide with each other even in a case of substantial coincidence in which there is a difference between the periodicity of the part change amount of the driver and the periodicity of the convulsion within a preset allowable range.

When detecting a convulsion of the driver, the convulsion detecting unit 105 outputs information indicating that a convulsion of the driver is detected (hereinafter, referred to as “convulsion occurrence information”) to the determination unit 106. For example, the convulsion occurrence information may include information regarding the part change amount of the driver in time series.

When not detecting a convulsion of the driver, the convulsion detecting unit 105 outputs information indicating that a convulsion of the driver is not detected (hereinafter, referred to as “convulsion non-occurrence information”) to the determination unit 106.

The determination unit 106 determines whether or not the driver has a physical condition abnormality on the basis of whether or not the posture collapse detecting unit 104 has detected posture collapse of the driver and whether or not the convulsion detecting unit 105 has detected a convulsion of the driver. That is, the determination unit 106 determines whether or not the driver has a physical condition abnormality on the basis of whether or not the posture collapse occurrence information has been output from the posture collapse detecting unit 104 and whether or not the convulsion occurrence information has been output from the convulsion detecting unit 105.

Specifically, the determination unit 106 determines whether or not the driver has a physical condition abnormality on the basis of a preset condition (hereinafter, referred to as a “physical condition abnormality determining condition”). When the physical condition abnormality determining condition is satisfied, the determination unit 106 determines that the driver has a physical condition abnormality. When the physical condition abnormality determining condition is not satisfied, the determination unit 106 determines that the driver does not have a physical condition abnormality.

The physical condition abnormality determining condition is set in advance by an administrator or the like, and is stored in a place that can be referred to by the determination unit 106. The physical condition abnormality determining condition can be appropriately set. As the physical condition abnormality determining condition, a condition that makes it possible to determine a physical condition abnormality of the driver on the basis of whether or not posture collapse has been detected and whether or not a convulsion has been detected only needs to be set.

The physical condition abnormality determining condition will be described with specific examples.

For example, the following <Condition 1> or <Condition 2> is set as the physical condition abnormality determining condition.

### <Condition 1>

“Posture collapse has not been detected, and a convulsion has been detected.”

### <Condition 2>

“Both posture collapse and a convulsion have been detected.”

For example, it is assumed that <Condition 1> is set as the physical condition abnormality determining condition. In this case, the determination unit 106 determines that the driver has a physical condition abnormality when the posture collapse non-occurrence information is output from the posture collapse detecting unit 104 and the convulsion occurrence information is output from the convulsion detecting unit 105.

As described above, when the convulsion does not involve a large change in posture but the driver has the convulsion, it can be said that the driver has a physical condition abnormality. If the determination unit 106 determines whether or not the driver has a physical condition abnormality only on the basis of detection of posture collapse, there is a possibility that it is determined that the driver does not have a physical condition abnormality even when the driver has a convulsion.

Meanwhile, the determination unit 106 of the physical condition abnormality determination device 1 according to the first embodiment can determine a physical condition abnormality of the driver not involving a large change in posture that can be said to be posture collapse by determining the physical condition abnormality of the driver according to the physical condition abnormality determining condition of <Condition 1>.

Note that it is also conceivable that the driver suddenly has a physical condition abnormality such as bending forward or warping like a shrimp without having a convulsion.

Therefore, for example, the following <Condition 3> may be set as the physical condition abnormality determining condition.

### <Condition 3>

“A convulsion has been detected without detection of posture collapse or posture collapse has been detected without detection of a convulsion.”

When <Condition 3> is set as the physical condition abnormality determining condition, the determination unit 106 determines that the driver has a physical condition abnormality when either posture collapse or a convulsion is detected. The determination unit 106 can determine whether or not the driver has a physical condition abnormality in consideration of both a physical condition abnormality in which posture collapse involving a large change in posture occurs and a physical condition abnormality of the driver not involving a large change in posture that can be said to be posture collapse.

For example, when <Condition 2> is set as the physical condition abnormality determining condition, the determination unit 106 can more surely determine that the driver has a physical condition abnormality.

Note that the physical condition abnormality determining condition may include a plurality of conditions. When the physical condition abnormality determining condition includes a plurality of conditions, the determination unit 106 may determine whether or not the driver has a physical condition abnormality by combining the plurality of conditions.

When determining that the driver has a physical condition abnormality, the determination unit 106 outputs the physical condition abnormality determining information to the output unit 107.

Note that when determining that the driver does not have a physical condition abnormality, the determination unit 106 may output information indicating that the driver does not have a physical condition abnormality (hereinafter, referred to as “physical condition abnormality non-occurrence information”) to the output unit 107.

The output unit 107 outputs the physical condition abnormality determining information output from the determination unit 106 to the output device 3.

When the physical condition abnormality non-occurrence information is output from the determination unit 106, the output unit 107 may output the physical condition abnormality non-occurrence information to the output device 3.

In addition, the output unit 107 may store the physical condition abnormality determining information or the physical condition abnormality non-occurrence information in a storage device such as the storage unit.

Note that the determination unit 106 may have the function of the output unit 107.

Details of the calibration process performed by the physical condition abnormality determination device 1 according to the first embodiment will be described.

It is estimated that a driver is in a normal state for a constant period from start of traveling of a vehicle. Therefore, the physical condition abnormality determination device 1 performs the calibration process, and sets a head reference value and a part reference value on the basis of imaged images acquired in a constant period (hereinafter, referred to as “reference value setting time”) from start of traveling of the vehicle. The reference value setting time is, for example, 15 seconds.

Specifically, first, a control unit (not illustrated) of the physical condition abnormality determination device 1 determines whether or not the reference value setting time has elapsed since start of traveling of the vehicle. For example, the control unit only needs to acquire vehicle speed information from a vehicle speed sensor (not illustrated), and to determine that traveling of the vehicle has been started on the basis of the vehicle speed. When determining that traveling of the vehicle has been started, the control unit starts counting the reference value setting time.

When the reference value setting time elapses, the control unit outputs a head position calibration instruction to the head position detecting unit 102. In addition, when the reference value setting time elapses, the control unit outputs a skeleton coordinate point calibration instruction to the skeleton point detecting unit 103.

When the head position calibration instruction is output from the control unit, the head position detecting unit 102 acquires imaged images for the reference value setting time. The head position detecting unit 102 only needs to acquire the imaged images for the reference value setting time from the storage unit. Note that, for example, when power of the vehicle is turned on, the imaged image acquiring unit 101 starts acquisition of an imaged image and stores the imaged image in the storage unit.

Then, the head position detecting unit 102 detects a head position of the driver for each of the acquired imaged images, and sets a head reference value of the driver on the basis of the detected head position of the driver. Note that an example of a method for detecting the head position of the driver by the head position detecting unit 102 has been described, and thus redundant description is omitted. For example, the head position detecting unit 102 sets an average value, a mode value, or a median value of the detected head positions as the head reference value. The head position detecting unit 102 stores the set head reference value of the driver in a place that can be referred to by the posture collapse detecting unit 104. In addition, the head position detecting unit 102 notifies the control unit that setting of the head reference value has been completed.

Note that, here, the head position detecting unit 102 collectively detects the head positions of the driver for imaged images for the reference value setting time at a timing when the control unit determines that the reference value setting time has elapsed, but this is merely an example. For example, the control unit may notify the head position detecting unit 102 of start of traveling of the vehicle when traveling of the vehicle starts, and the head position detecting unit 102 may detect the head position of the driver every time an imaged image is output from the imaged image acquiring unit 101 when the head position detecting unit 102 is notified of the start of traveling of the vehicle by the control unit. The head position detecting unit 102 stores head position information regarding the detected head position of the driver in the storage unit in association with the detection date and time. The reference value setting time is counted by the head position detecting unit 102, and when determining that the reference value setting time has elapsed, the head position detecting unit 102 sets the head reference value of the driver using the head position information stored in the storage unit. The head reference value of the driver may be set by such a method.

When the skeleton coordinate point calibration instruction is output from the control unit, the skeleton point detecting unit 103 acquires imaged images for the reference value setting time. The skeleton point detecting unit 103 only needs to acquire the imaged images for the reference value setting time from the storage unit.

Then, the skeleton point detecting unit 103 detects a skeleton coordinate point of the driver for each of the acquired imaged images, and sets a part reference value of the driver on the basis of the detected skeleton coordinate point of the driver. Note that an example of a method for detecting the skeleton coordinate point of the driver by the skeleton point detecting unit 103 has been described, and thus redundant description is omitted. For example, the skeleton point detecting unit 103 sets an average value, a mode value, or a median value of the detected skeleton coordinate points as the part reference value of the driver. The skeleton point detecting unit 103 stores the set part reference value in a place that can be referred to by the convulsion detecting unit 105. The skeleton point detecting unit 103 detects the skeleton coordinate point and sets the part reference value of the driver for each skeleton coordinate point. When the set part reference value of the driver is stored, the skeleton point detecting unit 103 stores the part reference value and information capable of specifying a skeleton coordinate point corresponding to the part reference value in association with each other. In addition, the skeleton point detecting unit 103 notifies the control unit that setting of the part reference value has been completed.

Note that, here, the skeleton point detecting unit 103 collectively detects the skeleton coordinate points of the driver for imaged images for the reference value setting time at a timing when the control unit determines that the reference value setting time has elapsed, but this is merely an example. For example, the control unit may notify the skeleton point detecting unit 103 of start of traveling of the vehicle when traveling of the vehicle starts, and the skeleton point detecting unit 103 may detect the skeleton coordinate point of the driver every time an imaged image is output from the imaged image acquiring unit 101 when the skeleton point detecting unit 103 is notified of the start of traveling of the vehicle by the control unit. The skeleton point detecting unit 103 stores skeleton coordinate point information regarding the detected skeleton coordinate point of the driver in the storage unit in association with the detection date and time and information capable of specifying a body part corresponding to the skeleton coordinate point. The reference value setting time is counted by the skeleton point detecting unit 103, and when determining that the reference value setting time has elapsed, the skeleton point detecting unit 103 sets the part reference value of the driver using the skeleton coordinate point information stored in the storage unit. The part reference value of the driver may be set by such a method.

When the calibration process is completed, that is, when setting of the head reference value and the part reference value is completed, the control unit notifies components (the imaged image acquiring unit 101, the head position detecting unit 102, the skeleton point detecting unit 103, the posture collapse detecting unit 104, the convulsion detecting unit 105, the determination unit 106, and the output unit 107) of the physical condition abnormality determination device 1 that the calibration process has been completed.

An operation of the physical condition abnormality determination device 1 according to the first embodiment will be described.

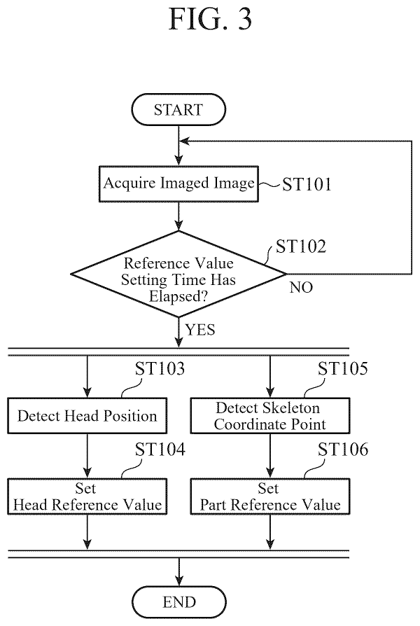

FIG. 3 is a flowchart for explaining an example of an operation of the calibration process performed by the physical condition abnormality determination device 1 according to the first embodiment.

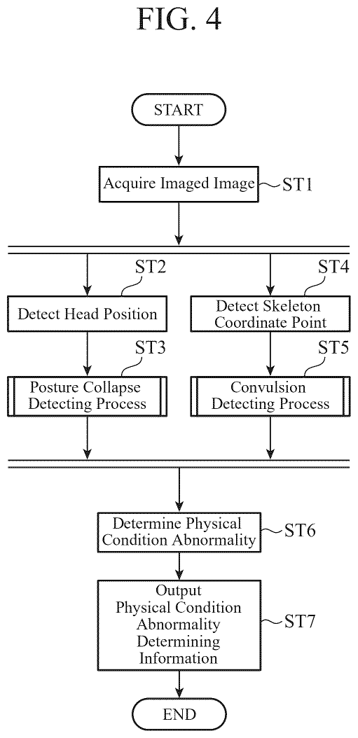

FIG. 4 is a flowchart for explaining an example of an operation of determining a physical condition abnormality of a driver, performed by the physical condition abnormality determination device 1 according to the first embodiment.

The operation illustrated in the flowchart of FIG. 3 is performed before the operation illustrated in the flowchart of FIG. 4 is performed.

First, an example of the operation of the calibration process performed by the physical condition abnormality determination device 1 will be described.

Note that, in FIG. 3, the processes of steps ST103 and ST104 are performed in parallel with the processes of steps ST105 and ST106. The operation illustrated in the flowchart of FIG. 3 is started, for example, when power of the vehicle is turned on.

The imaged image acquiring unit 101 acquires an imaged image from the imaging device 2 (step ST101).

The imaged image acquiring unit 101 outputs the acquired imaged image to the head position detecting unit 102 and the skeleton point detecting unit 103, and stores the imaged image in the storage unit.

The physical condition abnormality determination device 1 repeats the process of step ST101 until the control unit determines that the reference value setting time has elapsed since start of traveling of the vehicle (“NO” in step ST102).

If the control unit determines that the reference value setting time has elapsed since start of traveling of the vehicle (“YES” in step ST102), the control unit outputs a head position calibration instruction to the head position detecting unit 102. In addition, when the reference value setting time elapses, the control unit outputs a skeleton coordinate point calibration instruction to the skeleton point detecting unit 103.

When the head position calibration instruction is output from the control unit in step ST102, the head position detecting unit 102 acquires imaged images for the reference value setting time, and detects the head position of the driver for each of the acquired imaged images (step ST103).

The head position detecting unit 102 sets a head reference value of the driver on the basis of the head position of the driver detected in step ST103 (step ST104). The head position detecting unit 102 stores the set head reference value of the driver in a place that can be referred to by the posture collapse detecting unit 104. In addition, the head position detecting unit 102 notifies the control unit that setting of the head reference value has been completed.

When a skeleton coordinate point calibration instruction is output from the control unit in step ST102, the skeleton point detecting unit 103 acquires imaged images for the reference value setting time, and detects the skeleton coordinate point of the driver for each of the acquired imaged images (step ST105).

The skeleton point detecting unit 103 sets a part reference value of the driver on the basis of the skeleton coordinate point of the driver detected in step ST105 (step ST106). The skeleton point detecting unit 103 stores the set part reference value of the driver in a place that can be referred to by the convulsion detecting unit 105. In addition, the skeleton point detecting unit 103 notifies the control unit that setting of the part reference value has been completed.

Note that, for example, when the control unit notifies the head position detecting unit 102 of start of traveling of the vehicle and the head position detecting unit 102 detects the head position of the driver every time the imaged image is output, the process of step ST102 is performed by the head position detecting unit 102 after step ST103 in the flowchart of FIG. 3.

For example, when the control unit notifies the skeleton point detecting unit 103 of start of traveling of the vehicle and the skeleton point detecting unit 103 detects the skeleton coordinate point of the driver every time the imaged image is output, the process of step ST102 is performed by the skeleton point detecting unit 103 after step ST105 in the flowchart of FIG. 3.

Next, an example of an operation of determining a physical condition abnormality of the driver, performed by the physical condition abnormality determination device 1 will be described.

When the calibration process as illustrated in the flowchart of FIG. 3 is performed and there is a notification that the calibration process has been completed from the control unit, the physical condition abnormality determination device 1 performs the operation illustrated in the flowchart of FIG. 4.

Note that, in FIG. 4, the processes of steps ST2 and ST3 are performed in parallel with the processes of steps ST4 and ST5.

When there is a notification that the calibration process has been completed from the control unit, the operation illustrated in the flowchart of FIG. 4 is repeatedly performed, for example, until power of the vehicle is turned off.

The imaged image acquiring unit 101 acquires an imaged image from the imaging device 2 (step ST1).

The imaged image acquiring unit 101 outputs the acquired imaged image to the head position detecting unit 102 and the skeleton point detecting unit 103. In addition, the imaged image acquiring unit 101 stores the acquired imaged image in the storage unit in association with the acquisition date and time of the imaged image.

On the basis of the imaged image acquired by imaged image acquiring unit 101 in step ST1, the head position detecting unit 102 detects a head position of the driver in the imaged image (step ST2).

The head position detecting unit 102 outputs the head position information to the posture collapse detecting unit 104.

The posture collapse detecting unit 104 performs a posture collapse detecting process of detecting posture collapse of the driver on the basis of the head position information output from the head position detecting unit 102 in step ST2 (step ST3).

On the basis of the imaged image acquired by imaged image acquiring unit 101 in step ST1, the skeleton point detecting unit 103 detects a skeleton coordinate point indicating a body part of the driver on the imaged image (step ST4).

The skeleton point detecting unit 103 outputs the skeleton coordinate point information to the convulsion detecting unit 105.

The convulsion detecting unit 105 performs a convulsion detecting process of detecting a convulsion of the driver on the basis of the skeleton coordinate point information output from the skeleton point detecting unit 103 in step ST4 (step ST5).

The determination unit 106 determines whether or not the driver has a physical condition abnormality on the basis of whether or not the posture collapse detecting unit 104 has detected posture collapse of the driver in step ST3 and whether or not the convulsion detecting unit 105 has detected a convulsion of the driver in step ST5 (step ST6).

Specifically, when the physical condition abnormality determining condition is satisfied, the determination unit 106 determines that the driver has a physical condition abnormality. When the physical condition abnormality determining condition is not satisfied, the determination unit 106 determines that the driver does not have a physical condition abnormality.

When determining that the driver has a physical condition abnormality, the determination unit 106 outputs the physical condition abnormality determining information to the output unit 107. When determining that the driver does not have a physical condition abnormality, the determination unit 106 may output the physical condition abnormality non-occurrence information to the output unit 107.

The output unit 107 outputs the physical condition abnormality determining information output from the determination unit 106 in step ST6 to the output device 3 (step ST7).

When the physical condition abnormality non-occurrence information is output from the determination unit 106, the output unit 107 may output the physical condition abnormality non-occurrence information to the output device 3. In addition, the output unit 107 may store the physical condition abnormality determining information or the physical condition abnormality non-occurrence information in a storage device such as the storage unit.

Note that, for example, when the physical condition abnormality determination device 1 does not include the head position detecting unit 102, the process of step ST2 can be omitted in the flowchart of FIG. 4.

For example, when the physical condition abnormality determining condition has contents such as <Condition 3> described above, in step ST6, when the posture collapse occurrence information is output from the posture collapse detecting unit 104, the determination unit 106 may determine that <Condition 3> is satisfied even when neither the convulsion occurrence information nor the convulsion non-occurrence information is output from the convulsion detecting unit 105, and may cause the convulsion detecting unit 105 to end the convulsion detecting process. In addition, when the convulsion occurrence information is output from the convulsion detecting unit 105, the determination unit 106 can determine that <Condition 3> is satisfied even when neither the posture collapse occurrence information nor the posture collapse non-occurrence information is output from the posture collapse detecting unit 104, and can cause the posture collapse detecting unit 104 to end the posture collapse detecting process.

As described above, when it is determined whether or not the driver has a physical condition abnormality from either the result of the posture collapse detecting process or the result of the convulsion detecting process, the determination unit 106 can prevent the posture collapse detecting process or the convulsion detecting process that is no longer necessary from being performed.

Note that, in FIG. 1, an arrow from the determination unit 106 to the posture collapse detecting unit 104 and an arrow from the determination unit 106 to the convulsion detecting unit 105 are not illustrated.

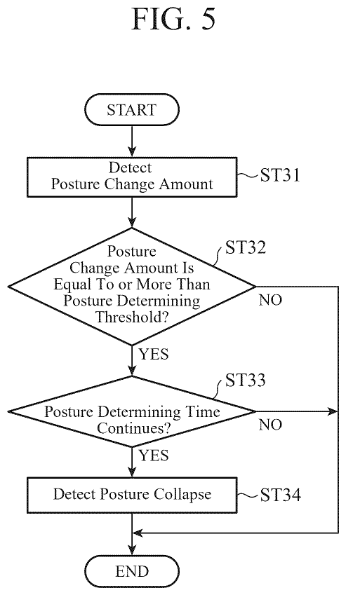

FIG. 5 is a flowchart for explaining a detailed operation of the posture collapse detecting process performed by the posture collapse detecting unit 104 in step ST3 of FIG. 4.

The posture collapse detecting unit 104 detects a posture change amount of the driver (step ST31).

The posture collapse detecting unit 104 determines whether or not the posture change amount of the driver detected in step ST31 is equal to or more than the posture determining threshold (step ST32).

In step ST32, if it is determined that the posture change amount of the driver is equal to or more than the posture determining threshold (“YES” in step ST32), the posture collapse detecting unit 104 determines whether or not the state in which the posture change amount of the driver detected in step ST32 is equal to or more than the posture determining threshold continues for the posture determining time (step ST33).

In step ST33, if it is determined that the state in which the posture change amount of the driver detected in step ST32 is equal to or more than the posture determining threshold continues for the posture determining time (“YES” in step ST33), the posture collapse detecting unit 104 detects posture collapse of the driver (step ST34). The posture collapse detecting unit 104 outputs the posture collapse occurrence information to the determination unit 106.

In step ST32, if it is determined that the posture change amount of the driver is less than the posture determining threshold (“NO” in step ST32), or in step ST33, if it is determined that the state in which the posture change amount of the driver detected in step ST32 is equal to or more than the posture determining threshold does not continue for the posture determining time (“NO” in step ST33), the posture collapse detecting unit 104 does not detect posture collapse of the driver. The posture collapse detecting unit 104 outputs the posture collapse non-occurrence information to the determination unit 106.

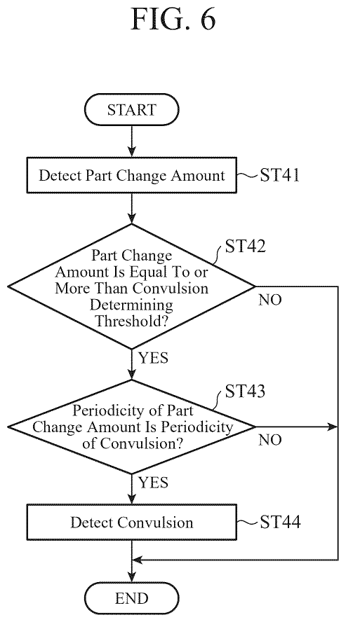

FIG. 6 is a flowchart for explaining a detailed operation of a convulsion detecting process performed by the convulsion detecting unit 105 in step ST5 of FIG. 4.

The convulsion detecting unit 105 detects a part change amount of the driver (step ST41). Note that the convulsion detecting unit 105 detects a part change amount which is a difference from a corresponding part reference value for each skeleton coordinate point in the step ST41.

The convulsion detecting unit 105 determines whether or not the part change amount of the driver detected in step ST41 is equal to or more than the convulsion determining threshold (step ST42).

In step ST42, if it is determined that the part change amount of the driver is equal to or more than the convulsion determining threshold (“YES” in step ST42), the convulsion detecting unit 105 determines whether or not periodicity of the part change amount of the driver detected in step ST41 is periodicity of the convulsion (step ST43).

In step ST43, if it is determined that the periodicity of the part change amount of the driver is the periodicity of convulsions (“YES” in step ST43), the convulsion detecting unit 105 detects a convulsion of the driver (step ST44). That is, the convulsion detecting unit 105 determines that the driver has a convulsion. The convulsion detecting unit 105 outputs the convulsion occurrence information to the determination unit 106.

In step ST42, if it is determined that the part change amount of the driver is less than the convulsion determining threshold (“NO” in step ST42), or in step ST43, if it is determined that the periodicity of the part change amount of the driver is not the periodicity of a convulsion (“NO” in step ST43), the convulsion detecting unit 105 does not detect a convulsion of the driver. The convulsion detecting unit 105 outputs the convulsion non-occurrence information to the determination unit 106.

Note that, in the flowchart of FIG. 6, processes are performed in the order of step ST42 and step ST43, but this is merely an example. The order of the process of step ST42 and the process of step ST43 may be reversed.

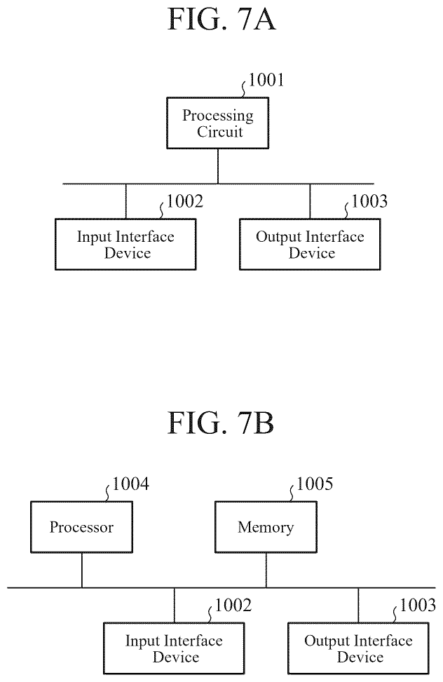

FIGS. 7A and 7B are diagrams illustrating an example of a hardware configuration of the physical condition abnormality determination device 1 according to the first embodiment.

In the first embodiment, functions of the imaged image acquiring unit 101, the head position detecting unit 102, the skeleton point detecting unit 103, the posture collapse detecting unit 104, the convulsion detecting unit 105, the determination unit 106, the output unit 107, and the control unit (not illustrated) are implemented by a processing circuit 1001. That is, the physical condition abnormality determination device 1 includes the processing circuit 1001 for performing control to determine whether or not the driver has a physical condition abnormality on the basis of an imaged image acquired from the imaging device 2.

The processing circuit 1001 may be dedicated hardware as illustrated in FIG. 7A, or a processor 1004 that executes a program stored in a memory as illustrated in FIG. 7B.

In a case where the processing circuit 1001 is dedicated hardware, for example, a single circuit, a composite circuit, a programmed processor, a parallel programmed processor, an application specific integrated circuit (ASIC), a field-programmable gate array (FPGA), or a combination thereof corresponds to the processing circuit 1001.

In a case where the processing circuit is the processor 1004, the functions of the imaged image acquiring unit 101, the head position detecting unit 102, the skeleton point detecting unit 103, the posture collapse detecting unit 104, the convulsion detecting unit 105, the determination unit 106, the output unit 107, and the control unit (not illustrated) are implemented by software, firmware, or a combination of software and firmware. Software or firmware is described as a program and stored in a memory 1005. The processor 1004 executes the functions of the imaged image acquiring unit 101, the head position detecting unit 102, the skeleton point detecting unit 103, the posture collapse detecting unit 104, the convulsion detecting unit 105, the determination unit 106, the output unit 107, and the control unit (not illustrated) by reading and executing the program stored in the memory 1005. That is, the physical condition abnormality determination device 1 includes the memory 1005 for storing a program that causes the above-described steps ST101 to ST106 illustrated in FIG. 3 and the above-described steps ST1 to ST7 illustrated in FIG. 4 to be executed as a result when the program is executed by the processor 1004. In addition, it can also be said that the program stored in the memory 1005 causes a computer to execute procedures or methods of processing performed by the imaged image acquiring unit 101, the head position detecting unit 102, the skeleton point detecting unit 103, the posture collapse detecting unit 104, the convulsion detecting unit 105, the determination unit 106, the output unit 107, and the control unit (not illustrated). Here, for example, a nonvolatile or volatile semiconductor memory such as RAM, read only memory (ROM), flash memory, erasable programmable read only memory (EPROM), or electrically erasable programmable read-only memory (EEPROM), a magnetic disk, a flexible disk, an optical disc, a compact disc, a mini disc, a digital versatile disc (DVD), or the like corresponds to the memory 1005.

Note that some of the functions of the imaged image acquiring unit 101, the head position detecting unit 102, the skeleton point detecting unit 103, the posture collapse detecting unit 104, the convulsion detecting unit 105, the determination unit 106, the output unit 107, and the control unit (not illustrated) may be implemented by dedicated hardware, and some of the functions may be implemented by software or firmware. For example, the function of the imaged image acquiring unit 101 can be implemented by the processing circuit 1001 as dedicated hardware, and the functions of the head position detecting unit 102, the skeleton point detecting unit 103, the posture collapse detecting unit 104, the convulsion detecting unit 105, the determination unit 106, the output unit 107, and the control unit (not illustrated) can be implemented by the processor 1004 reading and executing a program stored in the memory 1005.

In addition, the physical condition abnormality determination device 1 includes an input interface device 1002 and an output interface device 1003 that perform wired communication or wireless communication with a device such as the imaging device 2 or the output device 3.

The storage unit (not illustrated) is constituted by the memory 1005 or the like.

In the first embodiment described above, the physical condition abnormality determination device 1 determines whether or not the driver has a physical condition abnormality, but this is merely an example. The physical condition abnormality determination device 1 may determine whether or not an occupant other than the driver has a physical condition abnormality. The physical condition abnormality determination device 1 can also determine whether or not a plurality of occupants has a physical condition abnormality.

In the first embodiment described above, the occupant is assumed to be an occupant in a vehicle, but this is merely an example. For example, the occupant may be an occupant in a ship, a train, or the like. The physical condition abnormality determination device 1 can determine whether or not an occupant in various mobile objects has a physical condition abnormality.

In addition, in the first embodiment described above, the physical condition abnormality determination device 1 is an in-vehicle device mounted on a vehicle, and the imaged image acquiring unit 101, the head position detecting unit 102, the skeleton point detecting unit 103, the posture collapse detecting unit 104, the convulsion detecting unit 105, the determination unit 106, the output unit 107, and the control unit (not illustrated) are included in the in-vehicle device.

It is not limited to this, and some of the imaged image acquiring unit 101, the head position detecting unit 102, the skeleton point detecting unit 103, the posture collapse detecting unit 104, the convulsion detecting unit 105, the determination unit 106, the output unit 107, and the control unit (not illustrated) may be mounted on an in-vehicle device of a vehicle, and the others may be included in a server connected to the in-vehicle device via a network. In this manner, the in-vehicle device and the server may constitute a physical condition abnormality determining system.

In addition, all of the imaged image acquiring unit 101, the head position detecting unit 102, the skeleton point detecting unit 103, the posture collapse detecting unit 104, the convulsion detecting unit 105, the determination unit 106, the output unit 107, and the control unit (not illustrated) may be included in the server.

As described above, according to the first embodiment, the physical condition abnormality determination device 1 includes: the posture collapse detecting unit 104 that detects posture collapse of an occupant in a mobile object on the basis of a head position of the occupant in an imaged image obtained by imaging at least a face of the occupant, the head position being detected on the basis of the imaged image; the skeleton point detecting unit 103 that detects a skeleton coordinate point indicating a body part of the occupant on the imaged image on the basis of the imaged image; the convulsion detecting unit 105 that detects a convulsion of the occupant on the basis of skeleton coordinate point information regarding the skeleton coordinate point detected by the skeleton point detecting unit 103; and the determination unit 106 that determines that the occupant has a physical condition abnormality when the posture collapse of the occupant is not detected and the convulsion of the occupant is detected. Therefore, the physical condition abnormality determination device 1 can determine a physical condition abnormality of an occupant not involving a large change in posture.

### Second Embodiment

In the first embodiment, the physical condition abnormality determination device determines whether or not an occupant has a physical condition abnormality on the basis of whether or not posture collapse of the occupant has been detected and whether or not a convulsion of the occupant has been detected.

In the second embodiment, an embodiment will be described in which a physical condition abnormality determination device determines whether or not an occupant has a physical condition abnormality in consideration of whether or not a pulse of the occupant is abnormal in addition to whether or not posture collapse of the occupant has been detected and whether or not a convulsion of the occupant has been detected.

Note that, in the following second embodiment, the occupant is assumed to be a driver as in the first embodiment. However, this is merely an example, and the physical condition abnormality determination device according to the second embodiment can determine whether or not an occupant other than a driver has a physical condition abnormality. In addition, the physical condition abnormality determination device according to the second embodiment can also determine whether or not a plurality of occupants has a physical condition abnormality.

In addition, the physical condition abnormality determination device according to the second embodiment is assumed to be mounted on a vehicle similarly to the physical condition abnormality determination device according to the first embodiment.

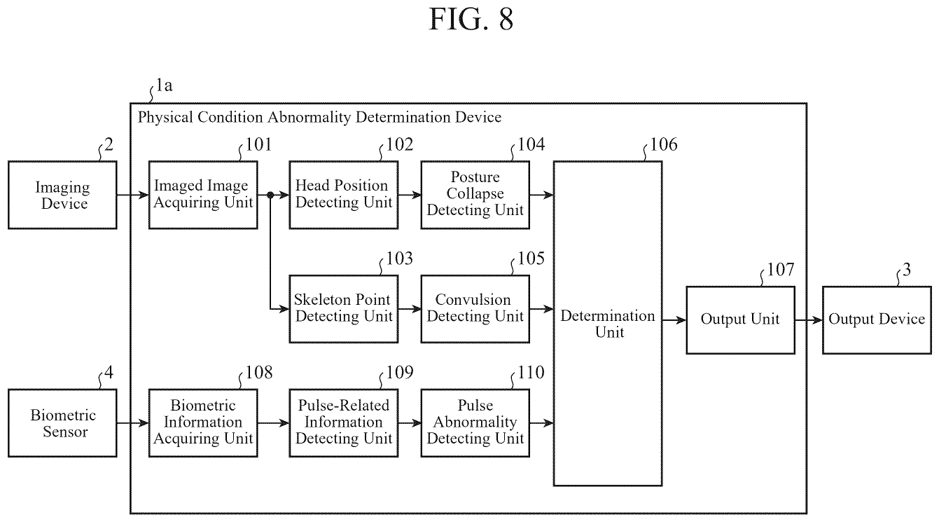

FIG. 8 is a diagram illustrating a configuration example of a physical condition abnormality determination device 1a according to the second embodiment.

In the configuration of the physical condition abnormality determination device 1a according to the second embodiment, the same components as those of the physical condition abnormality determination device 1 described with reference to FIG. 1 in the first embodiment are denoted by the same reference numerals, and redundant description is omitted.

Similarly to the physical condition abnormality determination device 1 according to the first embodiment, the physical condition abnormality determination device 1a does not necessarily include a head position detecting unit 102.

The physical condition abnormality determination device 1a according to the second embodiment is different from the physical condition abnormality determination device 1 according to the first embodiment in including a biometric information acquiring unit 108, a pulse-related information detecting unit 109, and a pulse abnormality detecting unit 110.

The physical condition abnormality determination device 1a is connected to a biometric sensor 4 in addition to an imaging device 2 and an output device 3.

The biometric sensor 4 is a device that measures biometric information such as a pulse or respiration of a driver. For example, the biometric sensor 4 is a radio wave biometric sensor using a microwave, disposed in the front of a vehicle interior, inside a seat belt, or inside a seat in a vehicle. For example, the biometric sensor 4 may be a wearable device worn by the driver himself or herself. For example, the biometric sensor 4 may be an imaging device. In a case where the biometric sensor 4 is an imaging device, the biometric sensor 4 and the imaging device 2 can be common.

Note that, in a case where the biometric sensor 4 is a wearable device, inconvenience for the driver may occur. By using a radio wave biometric sensor or the imaging device 2 disposed in the vehicle as the biometric sensor 4, it is possible to detect biometric information of the driver without causing inconvenience for the driver. In general, an imaging device for so-called PMS is mounted on the vehicle. When the imaging device 2 is used as the biometric sensor 4, it is not necessary to add a complicated device or the like for detecting biometric information of the driver.

The biometric information acquiring unit 108 acquires the biometric information of the driver from the biometric sensor 4.

The biometric information acquiring unit 108 outputs the acquired biometric information of the driver to the pulse-related information detecting unit 109.

The pulse-related information detecting unit 109 detects information regarding a pulse of the driver (hereinafter, referred to as “pulse-related information”) on the basis of the biometric information acquired by the biometric information acquiring unit 108.

In the second embodiment, the pulse-related information includes information regarding a pulse rate of the driver and information regarding a change amount of the pulse rate of the driver.

Note that in the second embodiment, the “pulse” also includes a “heartbeat”. For example, in a case where the biometric sensor 4 is the imaging device 2, the biometric information is an imaged image. For example, the pulse-related information detecting unit 109 can detect a pulse of the driver on the basis of a minute luminance change due to a flow of blood on a skin surface of the driver on the imaged image. Meanwhile, for example, in a case where the biometric sensor 4 is a radio wave biometric sensor such as a Doppler sensor, since the radio wave biometric sensor detects movement of a heart, what can be detected on the basis of the biometric information is not a pulse but a heartbeat.

The pulse-related information detecting unit 109 only needs to detect the pulse rate of the driver on the basis of the biometric information of the driver by a known method. For example, in a case where the biometric sensor 4 is the imaging device 2, the pulse-related information detecting unit 109 only needs to detect a pulse rate of the driver on the basis of biometric information of the driver (an imaged image in this case) using a known image recognition technique.

For example, the pulse-related information detecting unit 109 only needs to store the detected pulse rate of the driver in a storage unit in association with the detection date and time, and to calculate a change amount of the pulse rate of the driver on the basis of the stored pulse rate of the driver in time series. Note that the length of a past period for which the pulse rate is used for calculation of the change amount of the pulse rate is determined in advance.

For example, the pulse-related information detecting unit 109 calculates the change amount of the pulse rate of the driver using the following equations (1) and (2). Note that the following equations (1) and (2) are equations for calculating the change amount of the pulse rate of the driver from biometric information (an imaged image in this case) one frame before and current biometric information in a case where the biometric sensor 4 is the imaging device 2.

$${x}_{t} = ( 1 - α ) {x}_{t - 1} + α {y}_{t} \qquad ( 1 )$$

$${z}_{t} = {x}_{t} - {y}_{t} \qquad ( 2 )$$

In equation (1), t represents a frame number, xₜ represents a feature amount at time t, xₜ₋₁ represents a feature amount at time t−1, yₜ represents a pulse rate at time t, a represents an adjustment parameter, and x_0=0 and y_0=0 are satisfied. In addition, 0<α<1 is satisfied.

A change amount zₜ of the pulse rate of the driver is calculated from a difference between the feature amount xₜ at time t and the pulse rate yₜ at time t as in equation (2). Note that when the pulse rate of the driver rapidly increases, the change amount zₜ of the pulse rate of the driver becomes a negative value, and when the pulse rate of the driver rapidly decreases, the change amount zₜ of the pulse rate of the driver becomes a negative value.

Note that the above-described method for calculating the change amount of the pulse rate of the driver is merely an example. For example, the pulse-related information detecting unit 109 may calculate a difference between biometric information one frame before and current biometric information for biometric information for past several seconds such as ten seconds, and may set an average value of the calculated differences as the change amount of the pulse rate of the driver. The pulse-related information detecting unit 109 only needs to calculate the change amount of the pulse rate of the driver by an appropriately determined method.

The pulse-related information detecting unit 109 outputs the detected pulse-related information to the pulse abnormality detecting unit 110.

The pulse abnormality detecting unit 110 detects that a pulse of the driver is in an abnormal state on the basis of the pulse-related information of the driver detected by the pulse-related information detecting unit 109. In the second embodiment, the state in which the pulse is in an abnormal state is referred to as a “pulse abnormality”. In the second embodiment, a process of detecting a pulse abnormality of the driver, performed by the pulse abnormality detecting unit 110 is referred to as a “pulse abnormality detecting process”.

Here, the pulse abnormality detecting unit 110 detects a pulse abnormality of the driver when determining that the pulse rate of the driver or the change amount of the pulse rate of the driver is abnormal. That is, the pulse abnormality detecting unit 110 determines that the driver has a pulse abnormality when determining that the pulse rate of the driver or the change amount of the pulse rate of the driver is abnormal.

In general, when a person has a physical condition abnormality, it is assumed that the pulse rate increases unlike in a normal state. In addition, in general, when a person has a physical condition abnormality, it is assumed that the pulse rate rapidly changes. Therefore, the pulse abnormality detecting unit 110 detects a pulse abnormality of the driver on the basis of the pulse rate of the driver and the change amount of the pulse rate of the driver.

Note that, in the second embodiment, the pulse abnormality detecting unit 110 detects a pulse abnormality of the driver when determining that the pulse rate of the driver or the change amount of the pulse rate of the driver is abnormal, but this is merely an example. The pulse abnormality detecting unit 110 may detect a pulse abnormality of the driver when determining that both the pulse rate of the driver and the change amount of the pulse rate of the driver are abnormal.

In the pulse abnormality detecting process, for example, the pulse abnormality detecting unit 110 detects a pulse abnormality of the driver when the pulse rate of the driver is equal to or more than a preset threshold (hereinafter, referred to as a “pulse rate determining threshold”). Note that the pulse abnormality detecting unit 110 compares an absolute value of the pulse rate of the driver with the pulse rate determining threshold. The pulse rate determining threshold is set in advance by an administrator or the like, and is stored in a place that can be referred to by the pulse abnormality detecting unit 110.

As the pulse rate determining threshold, for example, a pulse rate that is generally determined to be life-threatening by a doctor is set. In general, a normal pulse rate of a person is said to be within a range of 60 bpm (Beats Per Minute) to 100 bpm, and a pulse rate that is generally determined to be life-threatening due to an acute change in physical condition of a person by a doctor is said to be equal to or more than 130 bpm (see, for example, Reference Literature 1).

### Reference Literature 1

Michael D Buist, director of intensive care unit, Gaye E Moore, research nurse, Stephen A Bernard, deputy director of intensive care unit, Bruce P Waxman, surgical programme director, Jeremy N Anderson, associate professor, and Tuan V Nguyen, senior fellow, “Effects of a medical emergency team on reduction of incidence of and mortality from unexpected cardiac arrests in hospital: preliminary study”, BMJ. 2002 Feb. 16, 324 (7334): 387-390, doi: 10.1136/bmj.324.7334.387

Therefore, for example, 130 bpm is set as the pulse rate determining threshold.

Note that in addition to the physical condition abnormality, for example, the pulse rate of a person may be increased by mental tension due to speech or the like, drinking, and exercise such as jogging. However, even when the pulse rate of a person is increased due to these reasons, the pulse rates after the increase are assumed to be about 100 bpm, 100 bpm, and 120 bpm on average, respectively, and are less likely to be equal to or more than 130 bpm.

For example, when the pulse rate of the driver is less than the pulse rate determining threshold, the pulse abnormality detecting unit 110 does not detect a pulse abnormality of the driver.

In addition, in the pulse abnormality detecting process, the pulse abnormality detecting unit 110 detects a pulse abnormality of the driver, for example, when a change amount of the pulse rate of the driver is equal to or more than a preset threshold (hereinafter, referred to as a “pulse change amount determining threshold”).

The pulse change amount determining threshold is set in advance by an administrator or the like, and is stored in a place that can be referred to by the pulse abnormality detecting unit 110. The administrator or the like sets a change amount of a pulse rate assumed to occur due to a physical condition abnormality of a person as the pulse change amount determining threshold.

Note that the pulse abnormality detecting unit 110 compares an absolute value of the change amount of the pulse rate of the driver with the pulse change amount determining threshold.

For example, when the change amount of the pulse rate of the driver is less than the pulse change amount determining threshold, the pulse abnormality detecting unit 110 does not detect a pulse abnormality of the driver.

When detecting a pulse abnormality of the driver, the pulse abnormality detecting unit 110 outputs information indicating that a pulse abnormality of the driver has been detected (hereinafter, referred to as “pulse abnormality occurrence information”) to the determination unit 106. For example, the pulse abnormality occurrence information may include information regarding the pulse rate of the driver and the change amount of the pulse rate of the driver.

When not detecting a pulse abnormality of the driver, the pulse abnormality detecting unit 110 outputs information indicating that a pulse abnormality of the driver has not been detected (hereinafter, referred to as “pulse abnormality non-occurrence information”) to the determination unit 106.

In the second embodiment, the determination unit 106 determines whether or not the driver has a physical condition abnormality on the basis of whether or not the posture collapse detecting unit 104 has detected posture collapse of the driver, whether or not the convulsion detecting unit 105 has detected a convulsion of the driver, and whether or not the pulse abnormality detecting unit 110 has detected a pulse abnormality of the driver. That is, the determination unit 106 determines whether or not the driver has a physical condition abnormality on the basis of whether or not the posture collapse occurrence information has been output from the posture collapse detecting unit 104, whether or not the convulsion occurrence information has been output from the convulsion detecting unit 105, and whether or not the pulse abnormality occurrence information has been output from the pulse abnormality detecting unit 110.

Therefore, in the second embodiment, as the physical condition abnormality determining condition, a condition for detecting a physical condition abnormality of the driver is set in consideration of a pulse abnormality in addition to posture collapse and a convulsion.

In the second embodiment, for example, the following <Condition 4>, <Condition 5>, <Condition 6>, or <Condition 7> is set as the physical condition abnormality determining condition.

### <Condition 4>

“Posture collapse has not been detected, and a convulsion and a pulse abnormality have been detected.”

### <Condition 5>

“Posture collapse and a pulse abnormality have not been detected, but a convulsion has been detected.”

### <Condition 6>

“Any one of posture collapse, a convulsion, and a pulse abnormality has been detected.”

### <Condition 7>

“All of posture collapse, a convulsion, and a pulse abnormality have been detected.”

For example, it is assumed that <Condition 4> is set as the physical condition abnormality determining condition. In this case, the determination unit 106 determines that the driver has a physical condition abnormality when the convulsion occurrence information is output from the convulsion detecting unit 105 and the pulse abnormality occurrence information is output from the pulse abnormality detecting unit 110 even when the posture collapse non-occurrence information is output from the posture collapse detecting unit 104.

As described above, when the convulsion does not involve a large change in posture but the driver has the convulsion, there is a high possibility that the driver has a physical condition abnormality. Here, by determining whether or not the driver has a pulse abnormality, the determination unit 106 can determine a physical condition abnormality of the driver with higher accuracy as compared with a case where the pulse abnormality is not considered.

For example, when <Condition 5> is set as the physical condition abnormality determining condition, the determination unit 106 can determine a physical condition abnormality of the driver not involving a large change in posture that can be said to be posture collapse.

Even when a seizure generally assumed to involve a large change in posture occurs, there may be an individual difference as to whether or not the seizure actually involves a large change in posture. For example, a person having a physical condition abnormality may hardly change his or her posture. For example, when <Condition 6> is set as the physical condition abnormality determining condition, the determination unit 106 can determine a physical condition abnormality of the driver in consideration of an individual difference that a person having a physical condition abnormality may hardly change his or her posture. When <Condition 6> is set as the physical condition abnormality determining condition, the determination unit 106 determines that the driver has a physical condition abnormality when the pulse abnormality occurrence information is output from the pulse abnormality detecting unit 110 even when the posture collapse non-occurrence information is output from the posture collapse detecting unit 104 and the convulsion non-occurrence information is output from the convulsion detecting unit 105. For example, when an epileptic seizure occurs, it is generally said that the epileptic seizure involves a convulsion and a pulse rate rapidly increases, but no convulsion is involved in some people. By determining that the driver has a physical condition abnormality when <Condition 6> is satisfied, the determination unit 106 can determine a physical condition abnormality of the driver that cannot be determined only from body movement, and can improve accuracy for determining the physical condition abnormality of the driver.

For example, when <Condition 7> is set as the physical condition abnormality determining condition, the determination unit 106 can more surely determine that the driver has a physical condition abnormality.

Note that, also in the second embodiment, the physical condition abnormality determining condition may include a plurality of conditions. When the physical condition abnormality determining condition includes a plurality of conditions, the determination unit 106 may determine whether or not the driver has a physical condition abnormality by combining the plurality of conditions.

When determining that the driver has a physical condition abnormality, the determination unit 106 outputs the physical condition abnormality determining information to the output unit 107. When determining that the driver does not have a physical condition abnormality, the determination unit 106 may output the physical condition abnormality non-occurrence information to the output unit 107.

In addition, the physical condition abnormality determination device 1a according to the second embodiment also performs a calibration process similarly to the physical condition abnormality determination device 1 according to the first embodiment. Since details of the calibration process have been described, redundant description is omitted.

An operation of the physical condition abnormality determination device 1a according to the second embodiment will be described.

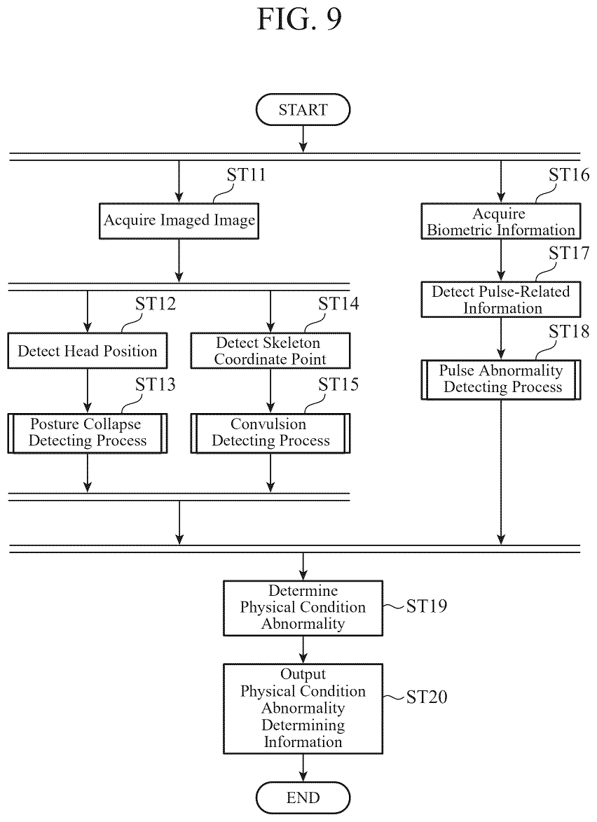

FIG. 9 is a flowchart for explaining an example of an operation of determining a physical condition abnormality of a driver, performed by the physical condition abnormality determination device 1a according to the second embodiment.

Note that the physical condition abnormality determination device 1a performs the calibration process before performing the operation illustrated in the flowchart of FIG. 9. That is, before the operation illustrated in the flowchart of FIG. 9 is performed, the operation described with reference to the flowchart of FIG. 3 in the first embodiment is performed.

When the calibration process is performed and there is a notification that the calibration process has been completed from the control unit, the physical condition abnormality determination device 1a performs the operation illustrated in the flowchart of FIG. 9.

Note that, in FIG. 9, the processes of steps ST11 to ST15 are performed in parallel with the processes of steps ST16 to ST18, and in FIG. 9, the processes of steps ST12 and ST13 are performed in parallel with the processes of steps ST14 and ST15.

When there is a notification that the calibration process has been completed from the control unit, the operation illustrated in the flowchart of FIG. 9 is repeatedly performed, for example, until power of the vehicle is turned off.

Since specific contents of the processes of steps ST11 to ST15 and step ST20 in FIG. 9 are similar to those of steps ST1 to ST5 and step ST7 in FIG. 4 described in the first embodiment, respectively, redundant description is omitted.

The biometric information acquiring unit 108 acquires biometric information of the driver from the biometric sensor 4 (step ST16).

The biometric information acquiring unit 108 outputs the acquired biometric information of the driver to the pulse-related information detecting unit 109.

The pulse-related information detecting unit 109 detects pulse-related information of the driver on the basis of the biometric information acquired by the biometric information acquiring unit 108 in step ST16 (step ST17).

The pulse-related information detecting unit 109 outputs the detected pulse-related information of the driver to the pulse abnormality detecting unit 110.

The pulse abnormality detecting unit 110 performs a pulse abnormality detecting process on the basis of the pulse-related information of the driver detected by the pulse-related information detecting unit 109 in step ST17 (step ST18).

When detecting a pulse abnormality of the driver, the pulse abnormality detecting unit 110 outputs pulse abnormality occurrence information to the determination unit 106. When not detecting a pulse abnormality of the driver, the pulse abnormality detecting unit 110 outputs pulse abnormality non-occurrence information to the determination unit 106.

The determination unit 106 determines whether or not the driver has a physical condition abnormality on the basis of whether or not the posture collapse detecting unit 104 has detected posture collapse of the driver in step ST13, whether or not the convulsion detecting unit 105 has detected a convulsion of the driver in step ST15, and whether or not the pulse abnormality detecting unit 110 has detected a pulse abnormality of the driver in step ST18 (step ST19).

Specifically, when the physical condition abnormality determining condition is satisfied, the determination unit 106 determines that the driver has a physical condition abnormality. When the physical condition abnormality determining condition is not satisfied, the determination unit 106 determines that the driver does not have a physical condition abnormality.

When determining that the driver has a physical condition abnormality, the determination unit 106 outputs the physical condition abnormality determining information to the output unit 107. When determining that the driver does not have a physical condition abnormality, the determination unit 106 may output the physical condition abnormality non-occurrence information to the output unit 107.

Note that, for example, when the physical condition abnormality determination device 1a does not include the head position detecting unit 102, the process of step ST12 can be omitted in the flowchart of FIG. 9.

For example, in a case where the physical condition abnormality determining condition has contents such as <Condition 6> described above, in step ST19, for example, when the posture collapse occurrence information is output from the posture collapse detecting unit 104, the determination unit 106 may determine that <Condition 6> is satisfied even when neither the convulsion occurrence information nor the convulsion non-occurrence information is output from the convulsion detecting unit 105 and neither the pulse abnormality occurrence information nor the pulse abnormality non-occurrence information is output from the pulse abnormality detecting unit 110, and may cause the convulsion detecting unit 105 to end the convulsion detecting process and may cause the pulse abnormality detecting unit 110 to end the pulse abnormality detecting process.

As described above, when it is determined whether or not the driver has a physical condition abnormality from any one of the result of the posture collapse detecting process, the result of the convulsion detecting process, and the result of the pulse abnormality detecting process, the determination unit 106 can prevent the posture collapse detecting process, the convulsion detecting process, or the pulse abnormality detecting process that is no longer necessary from being performed.

Note that, in FIG. 8, an arrow from the determination unit 106 to the posture collapse detecting unit 104, an arrow from the determination unit 106 to the convulsion detecting unit 105, and an arrow from the determination unit 106 to the pulse abnormality detecting unit 110 are not illustrated.

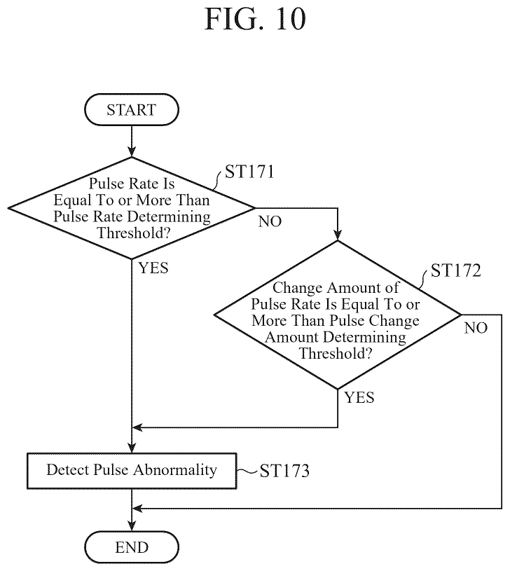

FIG. 10 is a flowchart for explaining a detailed operation of a pulse abnormality detecting process performed by the pulse abnormality detecting unit 110 in step ST18 of FIG. 9.

The pulse abnormality detecting unit 110 determines whether or not the pulse rate of the driver is equal to or more than the pulse rate determining threshold on the basis of the pulse-related information of the driver output from the pulse-related information detecting unit 109 in step ST17 of FIG. 9 (step ST171).

In step ST171, if it is determined that the pulse rate of the driver is less than the pulse rate determining threshold (“NO” in step ST171), the pulse abnormality detecting unit 110 determines whether or not a change amount of the pulse rate of the driver is equal to or more than the pulse change amount determining threshold on the basis of the pulse-related information of the driver output from the pulse-related information detecting unit 109 in step ST17 of FIG. 9 (step ST172).

In step ST171, if it is determined that the pulse rate of the driver is equal to or more than the pulse rate determining threshold (“YES” in step ST171), or in step ST172, if it is determined that the change amount of the pulse rate of the driver is equal to or more than the pulse change amount determining threshold (“YES” in step ST172), the pulse abnormality detecting unit 110 detects a pulse abnormality of the driver (step ST173). That is, the pulse abnormality detecting unit 110 determines that the driver has a pulse abnormality. The pulse abnormality detecting unit 110 outputs the pulse abnormality occurrence information to the determination unit 106.

In step ST172, if it is determined that the change amount of the pulse rate of the driver is less than the pulse change amount determining threshold (“NO” in step ST172), the pulse abnormality detecting unit 110 does not detect a pulse abnormality of the driver. That is, the pulse abnormality detecting unit 110 determines that the driver does not have a pulse abnormality. The pulse abnormality detecting unit 110 outputs the pulse abnormality non-occurrence information to the determination unit 106.

Note that, in the flowchart of FIG. 10, processes are performed in the order of step ST171 and step ST172, but this is merely an example. The order of the process of step ST171 and the process of step ST172 may be reversed.

For example, when the pulse abnormality detecting unit 110 detects a pulse abnormality of the driver by determining that both the pulse rate of the driver and the change amount of the pulse rate of the driver are abnormal, in the operation illustrated in the flowchart of FIG. 10, the process of step ST172 is performed if “YES” is obtained in the process of step ST171.

Since a hardware configuration of the physical condition abnormality determination device 1a according to the second embodiment is similar to the hardware configuration of the physical condition abnormality determination device 1 described with reference to FIGS. 7A and 7B in the first embodiment, description thereof is omitted.

In the second embodiment, functions of the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, and the control unit (not illustrated) are implemented by a processing circuit 1001. That is, the physical condition abnormality determination device 1a includes the processing circuit 1001 for performing control to determine whether or not the driver has a physical condition abnormality on the basis of an imaged image acquired from the imaging device 2 and biometric information acquired from the biometric sensor 4.

The processing circuit 1001 executes functions of the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, and the control unit (not illustrated) by reading and executing a program stored in a memory 1005. That is, the physical condition abnormality determination device 1a includes the memory 1005 for storing a program that causes the above-described steps ST101 to ST106 illustrated in FIG. 3 and the above-described steps ST11 to ST20 illustrated in FIG. 9 to be executed as a result when the program is executed by the processing circuit 1001. In addition, it can also be said that the program stored in the memory 1005 causes a computer to execute procedures or methods performed by the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, and the control unit (not illustrated).

The physical condition abnormality determination device 1a includes an input interface device 1002 and an output interface device 1003 that perform wired communication or wireless communication with a device such as the imaging device 2, the output device 3, or the biometric sensor 4.

The storage unit (not illustrated) is constituted by the memory 1005 or the like.

Note that, in the second embodiment described above, the occupant is assumed to be an occupant in the vehicle, but this is merely an example. For example, the occupant may be an occupant in a ship, a train, or the like. The physical condition abnormality determination device 1a can determine whether or not an occupant in various mobile objects has a physical condition abnormality.

In addition, in the second embodiment described above, the physical condition abnormality determination device 1a is an in-vehicle device mounted on a vehicle, and the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, and the control unit (not illustrated) are included in the in-vehicle device.

It is not limited to this, and some of the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, and the control unit (not illustrated) may be mounted on an in-vehicle device of a vehicle, and the others may be included in a server connected to the in-vehicle device via a network. In this manner, the in-vehicle device and the server may constitute a physical condition abnormality determining system.

In addition, all of the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, and the control unit (not illustrated) may be included in the server.

As described above, according to the second embodiment, the physical condition abnormality determination device 1a includes: the biometric information acquiring unit 108 that acquires biometric information of an occupant; the pulse-related information detecting unit 109 that detects pulse-related information regarding a pulse of the occupant on the basis of the biometric information acquired by the biometric information acquiring unit 108; and the pulse abnormality detecting unit 110 that detects a pulse abnormality of the occupant on the basis of the pulse-related information of the occupant detected by the pulse-related information detecting unit 109, and the determination unit 106 determines that the occupant has a physical condition abnormality when posture collapse of the occupant is not detected, a convulsion of the occupant is detected, and a pulse abnormality of the occupant is detected. Therefore, therefore, the physical condition abnormality determination device 1a can determine a physical condition abnormality of an occupant not involving a large change in posture, and can determine a physical condition abnormality of the occupant that cannot be determined only from body movement, and can improve accuracy for determining the physical condition abnormality of the occupant.

### Third Embodiment

In the second embodiment, the physical condition abnormality determination device determines whether or not an occupant has a physical condition abnormality in consideration of whether or not a pulse of the occupant is abnormal in addition to whether or not posture collapse of the occupant has been detected and whether or not a convulsion of the occupant has been detected.

Here, when the occupant has a convulsion, there is a problem that a large amount of artifact noise is included in the biometric information of the occupant due to body shake of the occupant by an increase in the degree of the convulsion. When the pulse abnormality detecting process is performed on the basis of biometric information including a large amount of artifact noise, erroneous detection of a pulse abnormality of the occupant may occur. The erroneous detection of the pulse abnormality of the occupant leads to erroneous determination of a physical condition abnormality of the occupant.

Therefore, in the third embodiment, when the physical condition abnormality of the occupant is determined, it is determined whether or not biometric information is used for determining the physical condition abnormality of the occupant in consideration of the degree of convulsion.

Note that, in the following third embodiment, the occupant is assumed to be a driver as in the second embodiment. However, this is merely an example, and the physical condition abnormality determination device according to the third embodiment can determine whether or not an occupant other than a driver has a physical condition abnormality. In addition, the physical condition abnormality determination device according to the third embodiment can also determine whether or not a plurality of occupants has a physical condition abnormality.

In addition, the physical condition abnormality determination device according to the third embodiment is assumed to be mounted on a vehicle similarly to the physical condition abnormality determination device according to the second embodiment.

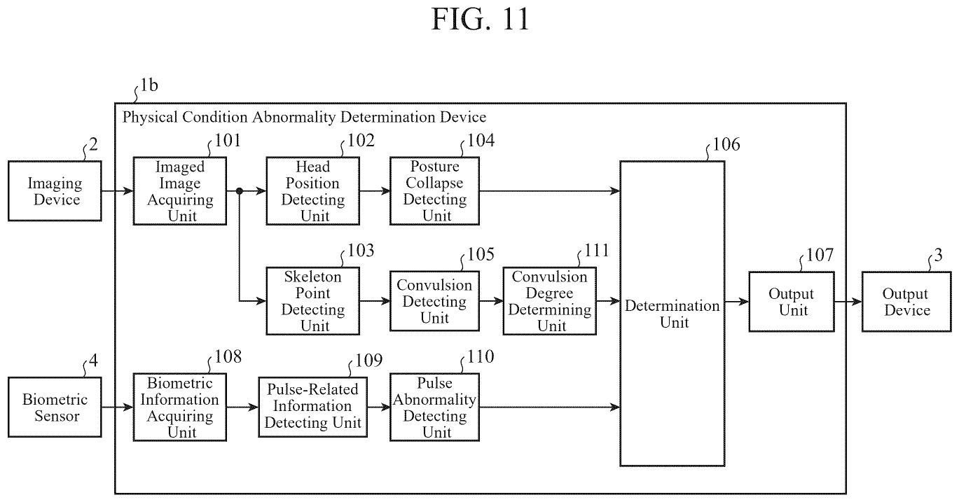

FIG. 11 is a diagram illustrating a configuration example of a physical condition abnormality determination device 1b according to the third embodiment.

In the configuration of the physical condition abnormality determination device 1b according to the third embodiment, the same components as those of the physical condition abnormality determination device 1a described with reference to FIG. 8 in the second embodiment are denoted by the same reference numerals, and redundant description is omitted.

Similarly to the physical condition abnormality determination device 1a according to the second embodiment, the physical condition abnormality determination device 1b does not necessarily include a head position detecting unit 102.

The physical condition abnormality determination device 1b according to the third embodiment is different from the physical condition abnormality determination device 1a according to the second embodiment in including a convulsion degree determining unit 111.

When the convulsion detecting unit 105 detects a convulsion of the driver, the convulsion degree determining unit 111 performs a convulsion degree determining process of determining the degree of convulsion (hereinafter, referred to as “convulsion degree”) of the driver on the basis of the convulsion occurrence information output from the convulsion detecting unit 105. Note that, in the third embodiment, the convulsion detecting unit 105 outputs convulsion occurrence information or convulsion non-occurrence information to the convulsion degree determining unit 111.

For example, the convulsion degree determining unit 111 determines the convulsion degree by comparing an amplitude of a part change amount with a preset threshold (hereinafter, referred to as a “convulsion degree determining threshold”) on the basis of a part change amount of the driver in time series included in the convulsion occurrence information. For example, the convulsion degree determining unit 111 determines that the convulsion degree is “high” when the amplitude of the part change amount is equal to or more than the convulsion degree determining threshold, and determines that the convulsion degree is “low” when a maximum value of the amplitude of the part change amount is less than the convulsion degree determining threshold. The convulsion degree determining threshold is set in advance by an administrator or the like, and is stored in a place that can be referred to by the convulsion degree determining unit 111. For example, the administrator or the like sets 10 (cm) as the convulsion degree determining threshold.

For example, the convulsion degree determining unit 111 determines the convulsion degree for each part change amount. For example, when determining that the convulsion degree is “high” for one of the part change amounts, the convulsion degree determining unit 111 determines that the convulsion degree of the driver is “high”.

Note that the method for determining the convulsion degree as described above is merely an example. The convulsion degree determining unit 111 only needs to determine a convulsion degree with which it is possible to determine whether or not artifact noise is assumed to be included to such an extent that a pulse rate cannot be appropriately determined. For example, the convulsion degree determining unit 111 may determine the convulsion degree in a plurality of stages instead of two values of “high” and “low”. In this case, for example, for each of the part change amounts, the convulsion degree determining unit 111 sets a convulsion degree at a stage with the largest numerical value as the convulsion degree of the driver. Note that the larger a numerical value of a stage, the larger the convulsion degree.

In the third embodiment, as an example, it is assumed that the convulsion degree of the driver determined by the convulsion degree determining unit 111 is “high” or “low”.

The convulsion degree determining unit 111 outputs the convulsion occurrence information output from the convulsion detecting unit 105 and information indicating the determined convulsion degree to the determination unit 106.

In addition, when the convulsion non-occurrence information is output from the convulsion detecting unit 105, the convulsion degree determining unit 111 outputs the convulsion non-occurrence information as it is to the determination unit 106.

In the third embodiment, the determination unit 106 determines whether or not the driver has a physical condition abnormality on the basis of whether or not the posture collapse detecting unit 104 has detected posture collapse of the driver, whether or not the convulsion detecting unit 105 has detected a convulsion of the driver, and whether or not the pulse abnormality detecting unit 110 has detected a pulse abnormality of the driver. When the convulsion occurrence information is output from the convulsion detecting unit 105, the determination unit 106 determines whether or not the driver has a physical condition abnormality in consideration of the convulsion degree imparted to the convulsion occurrence information.

Therefore, in the third embodiment, as the physical condition abnormality determining condition, a condition for detecting a physical condition abnormality of the driver is set in consideration of the convulsion degree.

In the third embodiment, for example, the following <Condition 8> or <Condition 9> is set as the physical condition abnormality determining condition.

### <Condition 8>

“Posture collapse is not detected, and a convulsion and a pulse abnormality have been detected. Note that when the convulsion degree is high, it is determined that a physical condition abnormality occurs regardless of whether or not a pulse abnormality has been detected.”

### <Condition 9>

“All of posture collapse, a convulsion, and a pulse abnormality have been detected. Note that when the convulsion degree is high, it does not matter whether or not the pulse abnormality has been detected.”

For example, it is assumed that <Condition 8> is set as the physical condition abnormality determining condition. In this case, when the posture collapse non-occurrence information is output from the posture collapse detecting unit 104 and the convulsion occurrence information is output from the convulsion detecting unit 105, the determination unit 106 determines whether or not the convulsion degree imparted to the convulsion occurrence information is high.

When the convulsion degree is high, the determination unit 106 determines that the driver has a physical condition abnormality even when the pulse abnormality occurrence information is output from the pulse abnormality detecting unit 110, the pulse abnormality non-occurrence information is output from the pulse abnormality detecting unit 110, or neither the pulse abnormality occurrence information nor the pulse abnormality non-occurrence information is output from the pulse abnormality detecting unit 110 yet.

As described above, when the driver has a convulsion, a large amount of artifact noise is included in the biometric information of the driver due to body shake of the driver by an increase in the degree of the convulsion. The pulse abnormality detected from the biometric information including a large amount of artifact noise may be erroneous detection, and use of the pulse abnormality for determining a physical condition abnormality of the driver leads to erroneous determination of the physical condition abnormality of the driver. The determination unit 106 can prevent erroneous determination of a physical condition abnormality of the driver by ignoring detection of a pulse abnormality when the convulsion degree is high in consideration of the convulsion degree.

In a case where the convulsion degree is low, the determination unit 106 determines that <Condition 8> is satisfied and the driver has a physical condition abnormality when the pulse abnormality occurrence information is output from the pulse abnormality detecting unit 110, and determines that <Condition 8> is not satisfied and the driver does not have a physical condition abnormality when the pulse abnormality non-occurrence information is output from the pulse abnormality detecting unit 110.

For example, it is assumed that <Condition 9> is set as the physical condition abnormality determining condition. In this case, when the convulsion occurrence information is output from the convulsion detecting unit 105, the determination unit 106 determines whether or not the convulsion degree imparted to the convulsion occurrence information is high.

In a case where the convulsion degree is high, the determination unit 106 determines that the driver has a physical condition abnormality when the posture collapse occurrence information is output from the posture collapse detecting unit 104. The determination unit 106 does not consider whether the pulse abnormality occurrence information is output from the pulse abnormality detecting unit 110, whether the pulse abnormality non-occurrence information is output from the pulse abnormality detecting unit 110, or whether neither the pulse abnormality occurrence information nor the pulse abnormality non-occurrence information is output from the pulse abnormality detecting unit 110 yet.

Even in this manner, the determination unit 106 can prevent erroneous determination of a physical condition abnormality of the driver by ignoring detection of a pulse abnormality when the convulsion degree is high in consideration of the convulsion degree.

Note that, also in the third embodiment, the physical condition abnormality determining condition may include a plurality of conditions. When the physical condition abnormality determining condition includes a plurality of conditions, the determination unit 106 may determine whether or not the driver has a physical condition abnormality by combining the plurality of conditions.

When determining that the driver has a physical condition abnormality, the determination unit 106 outputs the physical condition abnormality determining information to the output unit 107. When determining that the driver does not have a physical condition abnormality, the determination unit 106 may output the physical condition abnormality non-occurrence information to the output unit 107.

In addition, the physical condition abnormality determination device 1b according to the third embodiment also performs a calibration process similarly to the physical condition abnormality determination device 1a according to the second embodiment. Since details of the calibration process have been described, redundant description is omitted.

An operation of the physical condition abnormality determination device 1b according to the third embodiment will be described.

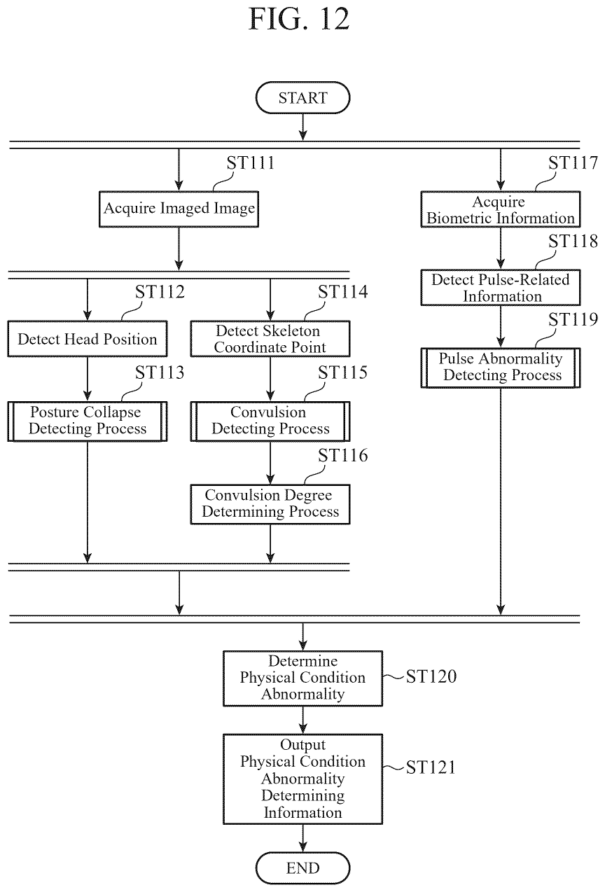

FIG. 12 is a flowchart for explaining an example of an operation of determining a physical condition abnormality of a driver, performed by the physical condition abnormality determination device 1b according to the third embodiment.

Note that the physical condition abnormality determination device 1b performs a calibration process before performing the operation illustrated in the flowchart of FIG. 12. That is, before the operation illustrated in the flowchart of FIG. 12 is performed, the operation described with reference to the flowchart of FIG. 3 in the first embodiment is performed.

When the calibration process is performed and there is a notification that the calibration process has been completed from the control unit, the physical condition abnormality determination device 1b performs the operation illustrated in the flowchart of FIG. 12.

Note that, in FIG. 12, the processes of steps ST111 to ST116 are performed in parallel with the processes of steps ST117 to ST119, and in FIG. 12, the processes of steps ST112 and ST113 are performed in parallel with the processes of steps ST114 to ST116.

When there is a notification that the calibration process has been completed from the control unit, the operation illustrated in the flowchart of FIG. 12 is repeatedly performed, for example, until power of the vehicle is turned off.

Since specific contents of the processes of steps ST111 to ST115, steps ST117 to ST119, and step ST121 in FIG. 12 are similar to those of steps ST11 to ST15, steps ST16 to ST18, and step ST20 in FIG. 9 described in the second embodiment, respectively, redundant description is omitted.

When the convulsion detecting unit 105 detects a convulsion of the driver in step ST115, the convulsion degree determining unit 111 performs a convulsion degree determining process of determining the convulsion degree of the driver on the basis of the convulsion occurrence information output from the convulsion detecting unit 105 (step ST116).

The convulsion degree determining unit 111 outputs the convulsion occurrence information output from the convulsion detecting unit 105 by adding information indicating the determined convulsion degree to the determination unit 106.

In addition, when the convulsion non-occurrence information is output from the convulsion detecting unit 105, the convulsion degree determining unit 111 outputs the convulsion non-occurrence information as it is to the determination unit 106.

The determination unit 106 determines whether or not the driver has a physical condition abnormality on the basis of whether or not the posture collapse detecting unit 104 has detected posture collapse of the driver in step ST113, whether or not the convulsion detecting unit 105 has detected a convulsion of the driver in step ST115, and whether or not the pulse abnormality detecting unit 110 has detected a pulse abnormality of the driver in step ST119 (step ST120). Note that the determination unit 106 determines whether or not the convulsion detecting unit 105 has detected a convulsion of the driver in step ST115 on the basis of whether or not the convulsion occurrence information has been output from the convulsion degree determining unit 111 in step ST116.

Specifically, when the physical condition abnormality determining condition is satisfied, the determination unit 106 determines that the driver has a physical condition abnormality. When the physical condition abnormality determining condition is not satisfied, the determination unit 106 determines that the driver does not have a physical condition abnormality.

When determining that the driver has a physical condition abnormality, the determination unit 106 outputs the physical condition abnormality determining information to the output unit 107. When determining that the driver does not have a physical condition abnormality, the determination unit 106 may output the physical condition abnormality non-occurrence information to the output unit 107.

Note that, for example, when the physical condition abnormality determination device 1b does not include the head position detecting unit 102, the process of step ST112 can be omitted in the flowchart of FIG. 12.

For example, in a case where the physical condition abnormality determining condition has contents such as <Condition 8> described above, in step ST20, for example, when the convulsion occurrence information is output from the convulsion degree determining unit 111 and information indicating that the convulsion degree is high is imparted to the convulsion occurrence information, the determination unit 106 may determine that <Condition 8> is satisfied even when neither the pulse abnormality occurrence information nor the pulse abnormality non-occurrence information is output from the pulse abnormality detecting unit 110, and may cause the pulse abnormality detecting unit 110 to end the pulse abnormality detecting process.

As described above, when it is determined whether or not the driver has a physical condition abnormality from any one of the result of the posture collapse detecting process, the result of the convulsion detecting process, and the result of the pulse abnormality detecting process, the determination unit 106 can prevent the posture collapse detecting process, the convulsion detecting process, or the pulse abnormality detecting process that is no longer necessary from being performed.

Note that, in FIG. 11, an arrow from the determination unit 106 to the posture collapse detecting unit 104, an arrow from the determination unit 106 to the convulsion detecting unit 105, and an arrow from the determination unit 106 to the pulse abnormality detecting unit 110 are not illustrated.

Since a hardware configuration of the physical condition abnormality determination device 1b according to the third embodiment is similar to the hardware configuration of the physical condition abnormality determination device 1 described with reference to FIGS. 7A and 7B in the first embodiment, drawings thereof are omitted.

In the third embodiment, functions of the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, the convulsion degree determining unit 111, and the control unit (not illustrated) are implemented by a processing circuit 1001. That is, the physical condition abnormality determination device 1b includes the processing circuit 1001 for performing control to determine whether or not the driver has a physical condition abnormality on the basis of an imaged image acquired from the imaging device 2 and biometric information acquired from the biometric sensor 4.

The processing circuit 1001 executes functions of the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, the convulsion degree determining unit 111, and the control unit (not illustrated) by reading and executing a program stored in a memory 1005. That is, the physical condition abnormality determination device 1b includes the memory 1005 for storing a program that causes the above-described steps ST101 to ST106 illustrated in FIG. 3 and the above-described steps ST111 to ST121 illustrated in FIG. 12 to be executed as a result when the program is executed by the processing circuit 1001. In addition, it can also be said that the program stored in the memory 1005 causes a computer to execute procedures or methods performed by the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, the convulsion degree determining unit 111, and the control unit (not illustrated).

The physical condition abnormality determination device 1b includes an input interface device 1002 and an output interface device 1003 that perform wired communication or wireless communication with a device such as the imaging device 2, the output device 3, or the biometric sensor 4.

The storage unit (not illustrated) is constituted by the memory 1005 or the like.

Note that, in the third embodiment described above, the occupant is assumed to be an occupant in the vehicle, but this is merely an example. For example, the occupant may be an occupant in a ship, a train, or the like. The physical condition abnormality determination device 1b can determine whether or not an occupant in various mobile objects has a physical condition abnormality.

In addition, in the third embodiment described above, the physical condition abnormality determination device 1b is an in-vehicle device mounted on a vehicle, and the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, the convulsion degree determining unit 111, and the control unit (not illustrated) are included in the in-vehicle device.

It is not limited to this, and some of the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, the convulsion degree determining unit 111, and the control unit (not illustrated) may be mounted on an in-vehicle device of a vehicle, and the others may be included in a server connected to the in-vehicle device via a network. In this manner, the in-vehicle device and the server may constitute a physical condition abnormality determining system.

In addition, all of the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, the convulsion degree determining unit 111, and the control unit (not illustrated) may be included in the server.

As described above, according to the third embodiment, the physical condition abnormality determination device 1b includes the convulsion degree determining unit 111 that determines the degree of a convulsion of an occupant detected by the convulsion detecting unit 105, and when posture collapse of the occupant is not detected and a convulsion of the occupant is detected, the determination unit 106 determines that the occupant has a physical condition abnormality regardless of whether or not a pulse abnormality of the occupant is detected when the degree of the convulsion of the occupant determined by the convulsion degree determining unit 111 is equal to or more than the convulsion degree determining threshold. Therefore, therefore, the physical condition abnormality determination device 1b can prevent erroneous determination of a physical condition abnormality of the occupant due to erroneous detection of a pulse abnormality.

### Fourth Embodiment

While a vehicle is traveling, vibration of the vehicle occurs in the vehicle. When the vibration of the vehicle occurs, a body of an occupant also shakes with the vibration of the vehicle.

In many vehicles, the vibration of the vehicle caused during traveling of the vehicle is an irregular vibration. In a physical condition abnormality determination device, a convulsion detecting unit does not erroneously detect a convulsion of the occupant when the body of the occupant shakes with the irregular vibration of the vehicle. This is because there is no periodicity in change in a part change amount.

However, for example, when the vehicle is traveling on a rumble strip or a mountain road, the vibration of the vehicle may be regular. When the vibration of the vehicle is regular, the body shake of the occupant caused by the vibration of the vehicle is also regular. In this case, the change in a part change amount has periodicity, the convulsion detecting unit may erroneously detect a convulsion of the occupant.

In a fourth embodiment, an embodiment will be described in which erroneous detection of a convulsion of an occupant is prevented when vibration of a vehicle is regular.

Note that, in the following fourth embodiment, the occupant is assumed to be a driver as in the second embodiment. However, this is merely an example, and the physical condition abnormality determination device according to the fourth embodiment can determine whether or not an occupant other than a driver has a physical condition abnormality. In addition, the physical condition abnormality determination device according to the fourth embodiment can also determine whether or not a plurality of occupants has a physical condition abnormality.

In addition, the physical condition abnormality determination device according to the fourth embodiment is assumed to be mounted on a vehicle similarly to the physical condition abnormality determination device according to the second embodiment.

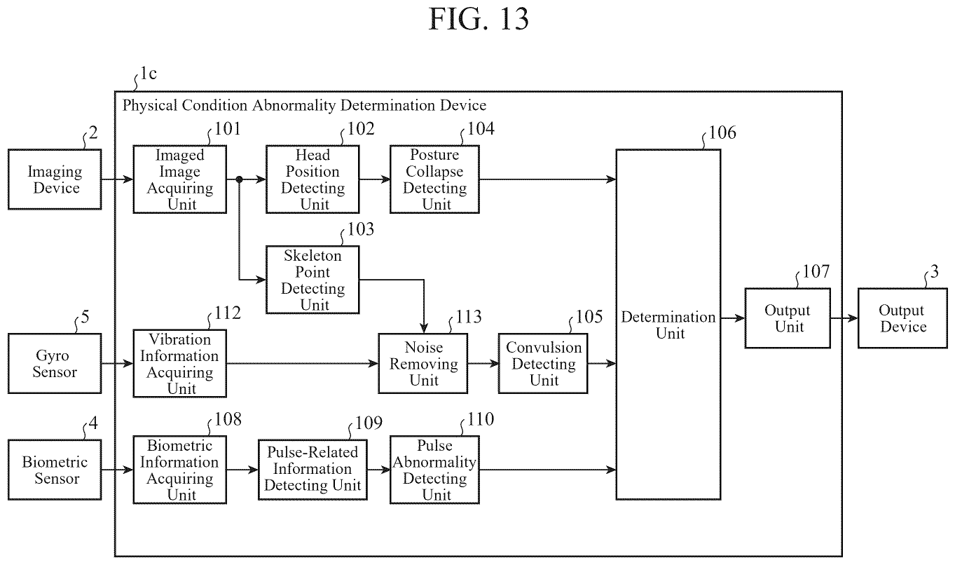

FIG. 13 is a diagram illustrating a configuration example of a physical condition abnormality determination device 1c according to the fourth embodiment.

In the configuration of the physical condition abnormality determination device 1c according to the fourth embodiment, the same components as those of the physical condition abnormality determination device 1a described with reference to FIG. 8 in the second embodiment are denoted by the same reference numerals, and redundant description is omitted.

Similarly to the physical condition abnormality determination device 1a according to the second embodiment, the physical condition abnormality determination device 1c does not necessarily include a head position detecting unit 102.

The physical condition abnormality determination device 1c according to the fourth embodiment is different from the physical condition abnormality determination device 1a according to the second embodiment in including a vibration information acquiring unit 112 and a noise removing unit 113.

The physical condition abnormality determination device 1c is connected to a gyro sensor 5 in addition to an imaging device 2, an output device 3, and a biometric sensor 4.

The gyro sensor 5 is mounted on a vehicle. Specifically, the gyro sensor 5 is, for example, an angular velocity sensor built in an in-vehicle device such as a car navigation device. The gyro sensor 5 can calculate a rotation angle per unit time (DEGREE PER SECOND). The gyro sensor 5 outputs vibration information generated on the basis of the calculated rotation angle to the physical condition abnormality determination device 1c. The vibration information includes information indicating vibration in an upward direction, a downward direction, a leftward direction, or a rightward direction of the vehicle.

The vibration information acquiring unit 112 acquires vibration information from the gyro sensor 5.

The vibration information acquiring unit 112 outputs the acquired vibration information to the noise removing unit 113.

In addition, the vibration information acquiring unit 112 stores the vibration information acquired from the gyro sensor 5 in a storage unit in association with the acquisition date and time.

The noise removing unit 113 performs a noise removing process of removing noise in skeleton point information of a driver output from a skeleton point detecting unit 103. Note that, in the fourth embodiment, noise refers to a component of movement of a skeleton coordinate point due to vibration of a vehicle, included in the skeleton coordinate point information of the driver.

An example of the noise removing process performed by the noise removing unit 113 will be described.

First, the noise removing unit 113 calculates movement of the vehicle, in other words, a time-series change in the position of the vehicle on the basis of the vibration information. The movement of the vehicle here is assumed to be at which timing and in which direction the vehicle is moving. Note that the noise removing unit 113 acquires vibration information for a preset time (hereinafter, referred to as “vibration determining time”) from the storage unit, and determines a time-series change in the movement of the vehicle in the vibration determining time. Note that the vibration determining time is a time having the same length as the length of a period when the convulsion detecting unit 105 determines periodicity of a part change amount.

In addition, the noise removing unit 113 calculates movement of a skeleton coordinate point of the driver, in other words, a time-series change in the position of the skeleton coordinate point of the driver on the basis of the skeleton coordinate point information of the driver. The movement of the skeleton coordinate point is assumed to be at which timing and in which direction the skeleton coordinate point on the image is moving. Note that the noise removing unit 113 acquires skeleton coordinate point information for the vibration determining time from the storage unit, and calculates a time-series change in a skeleton coordinate point of the driver in the vibration determining time.

Next, the noise removing unit 113 compares movement of the vehicle with movement of a skeleton coordinate point of the driver, and determines whether or not the movement of the vehicle is synchronized with the movement of the skeleton coordinate point of the driver. That is, the noise removing unit 113 determines whether or not the vehicle and the skeleton coordinate point of the driver are moving in the same direction at the same timing.

Since a position where the gyro sensor 5 is disposed, a detection range of the gyro sensor 5, a position where the imaging device 2 is disposed, and an angle of view of the imaging device 2 are known in advance, the noise removing unit 113 can associate the movement of the vehicle indicated by vibration information with the movement of the skeleton coordinate point indicated by an imaged image.

Note that the noise removing unit 113 determines, for each skeleton coordinate point, whether or not the movement of the skeleton coordinate point is synchronized with the movement of the vehicle.

Note that, in the fourth embodiment, “synchronization” is not limited to a case where the movements completely coincide with each other, and includes substantial synchronization in which an error within a preset allowable range occurs.

For example, when an error between the time-series change in the position of the vehicle and the time-series change in the position of the skeleton coordinate point of the driver is equal to or less than a preset threshold (for example, 5%), the noise removing unit 113 determines that the movement of the vehicle is synchronized with the movement of the skeleton coordinate point. The error referred to herein may be, for example, an average of errors between the time-series change in the position of the vehicle detected in the vibration determining time and the time-series change in the position of the skeleton coordinate point of the driver.

When the movement of the vehicle is synchronized with the movement of the skeleton coordinate point of the driver, in other words, when the position of the vehicle and the position of the skeleton coordinate point of the driver are moving in the same direction at the same timing, the noise removing unit 113 determines that the movement of the skeleton coordinate point is caused by vibration of the vehicle.

Then, the noise removing unit 113 obtains a difference between skeleton coordinate point information of the driver for the vibration determining time and vibration information for the vibration determining time. The noise removing unit 113 outputs the skeleton coordinate point information of the driver for the vibration determining time after obtaining the difference to the convulsion detecting unit 105. When the movement of the vehicle is synchronized with the movement of the skeleton coordinate point, the difference between the skeleton coordinate point information of the driver for the vibration determining time and the vibration information for the vibration determining time is almost zero.

Meanwhile, when the movement of the vehicle is not synchronized with the movement of the skeleton coordinate point of the driver, in other words, when the position of the vehicle and the position of the skeleton coordinate point of the driver are not moving in the same direction at the same timing, the noise removing unit 113 determines that the movement of the skeleton coordinate point of the driver is not caused by vibration of the vehicle. That is, the noise removing unit 113 determines that the movement of the skeleton coordinate point of the driver is highly likely to be caused by a convulsion of the driver.

The noise removing unit 113 outputs the skeleton coordinate point information of the driver for the vibration determining time to the convulsion detecting unit 105 as it is.

Note that, in the fourth embodiment, the convulsion detecting unit 105 detects a convulsion of the driver on the basis of the skeleton coordinate point information of the driver for the vibration determining time output from the noise removing unit 113.

Another example of the noise removing process performed by the noise removing unit 113 will be described.

In the example of the noise removing process described above, the noise removing unit 113 determines whether or not to perform noise removal depending on whether or not the movement of the vehicle is synchronized with the movement of the skeleton coordinate point of the driver, but this is merely an example. For example, the noise removing unit 113 may determine whether or not to perform noise removal depending on whether or not movement of a skeleton coordinate point of a passenger is synchronized with the movement of the skeleton coordinate point of the driver. In this case, the physical condition abnormality determination device 1c is not necessarily connected to the gyro sensor 5. The physical condition abnormality determination device 1c does not have to include the vibration information acquiring unit 112.

In this case, when not only the driver but also an occupant other than the driver is present in the vehicle interior on the basis of the imaged image acquired by the imaged image acquiring unit 101, the skeleton point detecting unit 103 detects a skeleton coordinate point indicating a body part of the occupant other than the driver on the imaged image. Note that, since the position where the imaging device 2 is disposed and the angle of view of the imaging device 2 are known, the skeleton point detecting unit 103 can specify which seat occupant the detected skeleton coordinate point belongs to. The skeleton point detecting unit 103 stores the detected skeleton coordinate point information in the storage unit in association with the detection date and time and information capable of specifying an occupant. Note that the information capable of specifying an occupant is, for example, information capable of specifying a seat on which the occupant is seated. In the following description, an occupant other than the driver is also referred to as a “passenger”.

First, the noise removing unit 113 calculates movement of a skeleton coordinate point of a passenger, in other words, a time-series change in the position of the skeleton coordinate point of the passenger on the basis of the skeleton coordinate point information. Note that the noise removing unit 113 acquires skeleton coordinate point information of the passenger for the vibration determining time from the storage unit, and calculates a time-series change in a skeleton coordinate point of the passenger in the vibration determining time.

In addition, the noise removing unit 113 calculates movement of a skeleton coordinate point of the driver, in other words, a time-series change in the position of the skeleton coordinate point of the driver on the basis of the skeleton coordinate point information.

Next, the noise removing unit 113 compares movement of a skeleton coordinate point of the passenger with movement of a skeleton coordinate point of the driver, and determines whether or not the movement of the skeleton coordinate point of the passenger is synchronized with the movement of the skeleton coordinate point of the driver. That is, the noise removing unit 113 determines whether or not the skeleton coordinate point of the passenger and the skeleton coordinate point of the driver are moving in the same direction at the same timing.

Note that the noise removing unit 113 determines, for each skeleton coordinate point, whether or not the movement of the skeleton coordinate point of the driver is synchronized with the movement of the skeleton coordinate point of the passenger.

When the movement of the skeleton coordinate point of the passenger is synchronized with the movement of the skeleton coordinate point of the driver, in other words, when the position of the skeleton coordinate point of the passenger and the position of the skeleton coordinate point of the driver are moving in the same direction at the same timing, the noise removing unit 113 determines that the movement of the skeleton coordinate point of the driver is caused by vibration of the vehicle.

Then, the noise removing unit 113 obtains a difference between skeleton coordinate point information of the driver for the vibration determining time and skeleton coordinate point information of the passenger for the vibration determining time. The noise removing unit 113 outputs the skeleton coordinate point information of the driver for the vibration determining time after obtaining the difference to the convulsion detecting unit 105. When the movement of the skeleton coordinate point of the passenger is synchronized with the movement of the skeleton coordinate point of the driver, a difference between the skeleton coordinate point information of the driver for the vibration determining time and the skeleton coordinate point information of the passenger for the vibration determining time is substantially zero.

Meanwhile, when the movement of the skeleton coordinate point of the passenger is not synchronized with the movement of the skeleton coordinate point of the driver, in other words, when the position of the skeleton coordinate point of the passenger and the position of the skeleton coordinate point of the driver are not moving in the same direction at the same timing, the noise removing unit 113 determines that the movement of the skeleton coordinate point of the driver is not caused by vibration of the vehicle. That is, the noise removing unit 113 determines that the movement of the skeleton coordinate point of the driver is highly likely to be caused by a convulsion of the driver.

The noise removing unit 113 outputs the skeleton coordinate point information of the driver for the vibration determining time to the convulsion detecting unit 105 as it is.

Note that the physical condition abnormality determination device 1c according to the fourth embodiment also performs a calibration process similarly to the physical condition abnormality determination device 1a according to the second embodiment. Since details of the calibration process have been described, redundant description is omitted.

An operation of the physical condition abnormality determination device 1c according to the fourth embodiment will be described.

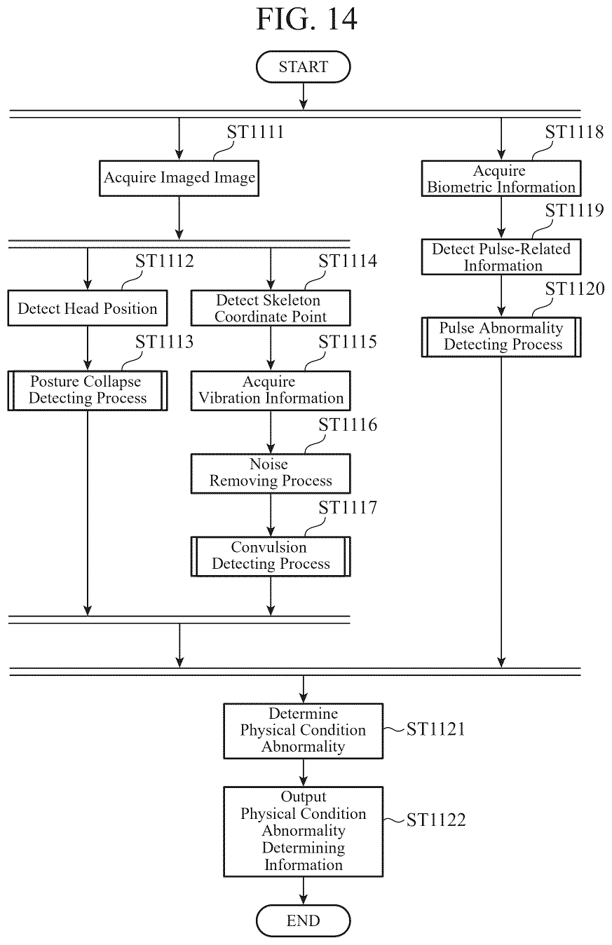

FIG. 14 is a flowchart for explaining an example of an operation of determining a physical condition abnormality of a driver, performed by the physical condition abnormality determination device 1c according to the fourth embodiment.

Note that the physical condition abnormality determination device 1c performs a calibration process before performing the operation illustrated in the flowchart of FIG. 14. That is, before the operation illustrated in the flowchart of FIG. 14 is performed, the operation described with reference to the flowchart of FIG. 3 in the first embodiment is performed.

In the operation illustrated in the flowchart of FIG. 14, in the physical condition abnormality determination device 1c, the noise removing unit 113 determines whether or not to perform noise removal depending on whether or not the movement of the vehicle is synchronized with the movement of the skeleton coordinate point of the driver.

When the calibration process is performed and there is a notification that the calibration process has been completed from the control unit, the physical condition abnormality determination device 1c performs the operation illustrated in the flowchart of FIG. 14.

Note that, in FIG. 14, the processes of steps ST1111 to ST1117 are performed in parallel with the processes of steps ST1118 to ST1120, and in FIG. 14, the processes of steps ST1112 and ST1113 are performed in parallel with the processes of steps ST1114 to ST1117.

When there is a notification that the calibration process has been completed from the control unit, the operation illustrated in the flowchart of FIG. 14 is repeatedly performed, for example, until power of the vehicle is turned off.

Since specific contents of the processes of steps ST1111 to ST1114, step ST1117, and steps ST1118 to ST1122 in FIG. 14 are similar to those of steps ST11 to ST15 and steps ST16 to ST20 in FIG. 9 described in the second embodiment, respectively, redundant description is omitted.

The vibration information acquiring unit 112 acquires vibration information from the gyro sensor 5 (step ST1115).

The vibration information acquiring unit 112 outputs the acquired vibration information to the noise removing unit 113.

In addition, the vibration information acquiring unit 112 stores the vibration information acquired from the gyro sensor 5 in a storage unit in association with the acquisition date and time.

The noise removing unit 113 performs a noise removing process of removing noise in the skeleton point information output from the skeleton point detecting unit 103 in step ST1115 (step ST1116).

In step ST1117, the convulsion detecting unit 105 detects a convulsion of the driver on the basis of the skeleton coordinate point information of the driver for the vibration determining time output from the noise removing unit 113 in step ST1116.

Note that, for example, when the physical condition abnormality determination device 1c does not include the head position detecting unit 102, the process of step ST1112 can be omitted in the flowchart of FIG. 14.

In the flowchart of FIG. 14, processes are performed in the order of step ST1114 and step ST1115, but this is merely an example. The order at which the process of step ST1114 is performed and the order at which the process of step ST1115 is performed may be reversed. In addition, the process of step ST1115 and the process of step ST1111 may be performed in parallel.

An example of the operation illustrated in the flowchart of FIG. 14 is an example of the operation of the physical condition abnormality determination device 1c in a case where the noise removing unit 113 determines whether or not to perform noise removal depending on whether or not the movement of the vehicle is synchronized with the movement of the skeleton coordinate point of the driver. For example, in a case where the noise removing unit 113 determines whether or not to perform noise removal depending on whether or not the movement of a skeleton coordinate point of a passenger is synchronized with the movement of the skeleton coordinate point of the driver, the operation of the physical condition abnormality determination device 1c can omit the process of step ST1115 in the flowchart of FIG. 14. In step ST1114, on the basis of the imaged image acquired by the imaged image acquiring unit 101 in step ST1111, the skeleton point detecting unit 103 detects a skeleton coordinate point indicating a body part of an occupant present in the vehicle interior on the imaged image.

Since a hardware configuration of the physical condition abnormality determination device 1c according to the fourth embodiment is similar to the hardware configuration of the physical condition abnormality determination device 1 described with reference to FIGS. 7A and 7B in the first embodiment, description thereof is omitted.

In the fourth embodiment, functions of the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, the vibration information acquiring unit 112, the noise removing unit 113, and the control unit (not illustrated) are implemented by a processing circuit 1001. That is, the physical condition abnormality determination device 1c includes the processing circuit 1001 for performing control to determine whether or not the driver has a physical condition abnormality on the basis of an imaged image acquired from the imaging device 2 and biometric information acquired from the biometric sensor 4.

The processing circuit 1001 executes functions of the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, the vibration information acquiring unit 112, the noise removing unit 113, and the control unit (not illustrated) by reading and executing a program stored in a memory 1005. That is, the physical condition abnormality determination device 1c includes the memory 1005 for storing a program that causes the above-described steps ST101 to ST106 illustrated in FIG. 3 and the above-described steps ST1111 to ST1122 illustrated in FIG. 14 to be executed as a result when the program is executed by the processing circuit 1001. In addition, it can also be said that the program stored in the memory 1005 causes a computer to execute procedures or methods performed by the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, the vibration information acquiring unit 112, the noise removing unit 113, and the control unit (not illustrated).

The physical condition abnormality determination device 1c includes an input interface device 1002 and an output interface device 1003 that perform wired communication or wireless communication with a device such as the imaging device 2, the output device 3, the biometric sensor 4, or the gyro sensor 5.

The storage unit (not illustrated) is constituted by the memory 1005 or the like.

Note that, in the fourth embodiment described above, the occupant is assumed to be an occupant in the vehicle, but this is merely an example. For example, the occupant may be an occupant in a ship, a train, or the like. The physical condition abnormality determination device 1c can determine whether or not an occupant in various mobile objects has a physical condition abnormality.

In addition, in the fourth embodiment described above, the physical condition abnormality determination device 1c is an in-vehicle device mounted on a vehicle, and the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, the vibration information acquiring unit 112, the noise removing unit 113, and the control unit (not illustrated) are included in the in-vehicle device.

It is not limited to this, and some of the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, the vibration information acquiring unit 112, the noise removing unit 113, and the control unit (not illustrated) may be mounted on an in-vehicle device of a vehicle, and the others may be included in a server connected to the in-vehicle device via a network. In this manner, the in-vehicle device and the server may constitute a physical condition abnormality determining system.

In addition, all of the imaged image acquiring unit 101, the head position detecting unit 102, the skeleton point detecting unit 103, the posture collapse detecting unit 104, the convulsion detecting unit 105, the determination unit 106, the output unit 107, the biometric information acquiring unit 108, the pulse-related information detecting unit 109, the pulse abnormality detecting unit 110, the vibration information acquiring unit 112, the noise removing unit 113, and the control unit (not illustrated) may be included in the server.

In the physical condition abnormality determination device 1c according to the fourth embodiment described above, the noise removing function is added to the physical condition abnormality determination device 1a according to the second embodiment, but this is merely an example. The contents of the fourth embodiment described above can be applied to the first embodiment and the third embodiment.

That is, the noise removing function can be added to the physical condition abnormality determination device 1 according to the first embodiment. In this case, in the configuration example of the physical condition abnormality determination device 1 illustrated in FIG. 1, the vibration information acquiring unit 112 and the noise removing unit 113 are added. In addition, the physical condition abnormality determination device 1 is connected to the gyro sensor 5. Note that the physical condition abnormality determination device 1 does not have to include the vibration information acquiring unit 112 and does not have to be connected to the gyro sensor 5.

The noise removing function can be added to the physical condition abnormality determination device 1b according to the third embodiment. In this case, in the configuration example of the physical condition abnormality determination device 1b illustrated in FIG. 8, the vibration information acquiring unit 112 and the noise removing unit 113 are added. In addition, the physical condition abnormality determination device 1b is connected to the gyro sensor 5. Note that the physical condition abnormality determination device 1b does not have to include the vibration information acquiring unit 112 and does not have to be connected to the gyro sensor 5.

As described above, according to the fourth embodiment, the physical condition abnormality determination device 1c includes: the vibration information acquiring unit 112 that acquires vibration information regarding vibration of a mobile object; and the noise removing unit 113 that removes noise included in skeleton coordinate point information of an occupant on the basis of the skeleton coordinate point information of the occupant and the vibration information, and the convulsion detecting unit 105 detects a convulsion of the occupant on the basis of the skeleton coordinate point information of the occupant after the noise removing unit 113 removes the noise. Therefore, the physical condition abnormality determination device 1c determines a physical condition abnormality of the occupant on the basis of the convulsion of the occupant detected in consideration of the vibration of the vehicle (mobile object), and therefore can improve accuracy for determining the physical condition abnormality of the occupant.

In addition, according to the fourth embodiment, in the physical condition abnormality determination device 1c, the skeleton point detecting unit 103 detects, in addition to a skeleton coordinate point of an occupant, a skeleton coordinate point indicating a body part of a passenger other than the occupant on an imaged image, the noise removing unit 113 that removes noise included in skeleton coordinate point information of the occupant on the basis of the skeleton coordinate point information of the occupant and skeleton coordinate point information of the passenger is included, and the convulsion detecting unit 105 detects a convulsion of the occupant on the basis of the skeleton coordinate point information of the occupant after the noise removing unit 113 removes the noise. Therefore, the physical condition abnormality determination device 1c determines a physical condition abnormality of the occupant on the basis of the convulsion of the occupant detected in consideration of the vibration of the vehicle (mobile object), and therefore can improve accuracy for determining the physical condition abnormality of the occupant.

In addition, the embodiments can be freely combined to each other, any constituent element in each of the embodiments can be modified, or any constituent element in each of the embodiments can be omitted.

### INDUSTRIAL APPLICABILITY

The physical condition abnormality determination device of the present disclosure can determine a physical condition abnormality of an occupant not involving a large change in posture.

### REFERENCE SIGNS LIST

1, 1a, 1b, 1c: physical condition abnormality determination device, 2: imaging device, 3: output device, 4: biometric sensor, 5: gyro sensor, 101: imaged image acquiring unit, 102: head position detecting unit, 103: skeleton point detecting unit, 104: posture collapse detecting unit, 105: convulsion detecting unit, 106: determination unit, 107: output unit, 108: biometric information acquiring unit, 109: pulse-related information detecting unit, 110: pulse abnormality detecting unit, 111: convulsion degree determining unit, 112: vibration information acquiring unit, 113: noise removing unit, 1001: processing circuit, 1002: input interface device, 1003: output interface device, 1004: processor, 1005: memory

1. A physical condition abnormality determination device comprising:
a processor; and
a memory storing a program to perform, upon executed by the processor, a process:
to detect posture collapse of an occupant in a mobile object on a basis of a head position of the occupant in an imaged image obtained by imaging at least a face of the occupant, the head position being detected on a basis of the imaged image;
to detect a skeleton coordinate point indicating a body part of the occupant on the imaged image on a basis of the imaged image;
to detect a convulsion of the occupant on a basis of skeleton coordinate point information regarding the skeleton coordinate point detected;
to determine that the occupant has a physical condition abnormality when the posture collapse of the occupant is not detected and the convulsion of the occupant is detected;
to acquire vibration information regarding vibration of the mobile object; and
to remove noise included in the skeleton coordinate point information of the occupant on a basis of the skeleton coordinate point information of the occupant and the vibration information, wherein
the process detects the convulsion of the occupant on a basis of the skeleton coordinate point information of the occupant after removing the noise.

2.-11. (canceled)

12. The physical condition abnormality determination device according to claim 1, wherein:
the process removes the noise by obtaining a difference between the skeleton coordinate point information of the occupant and the vibration information when movement of the occupant based on the skeleton coordinate point information of the occupant is synchronized with movement of the mobile object based on the vibration information.

13. A physical condition abnormality determination device comprising:
a processor; and
a memory storing a program to perform, upon executed by the processor, a process:
to detect posture collapse of an occupant in a mobile object on a basis of a head position of the occupant in an imaged image obtained by imaging at least a face of the occupant, the head position being detected on a basis of the imaged image;
to detect a skeleton coordinate point indicating a body part of the occupant on the imaged image on a basis of the imaged image;
to detect a convulsion of the occupant on a basis of skeleton coordinate point information regarding the skeleton coordinate point detected; and
to determine that the occupant has a physical condition abnormality when the posture collapse of the occupant is not detected and the convulsion of the occupant is detected, wherein
the process detects, in addition to the skeleton coordinate point of the occupant, the skeleton coordinate point indicating a body part of a passenger other than the occupant on the imaged image, further comprising:
to remove noise included in the skeleton coordinate point information of the occupant on a basis of the skeleton coordinate point information of the occupant and the skeleton coordinate point information of the passenger, and
the process detects the convulsion of the occupant on a basis of the skeleton coordinate point information of the occupant after removing the noise.

14. The physical condition abnormality determination device according to claim 13, wherein:
the process removes the noise by obtaining a difference between the skeleton coordinate point information of the occupant and the skeleton coordinate point information of the passenger when movement of the occupant based on the skeleton coordinate point information of the occupant is synchronized with movement of the passenger based on the skeleton coordinate point information of the passenger.

15. A physical condition abnormality determination device comprising:
a processor; and
a memory storing a program to perform, upon executed by the processor, a process:
to detect posture collapse of an occupant in a mobile object on a basis of a head position of the occupant in an imaged image obtained by imaging at least a face of the occupant, the head position being detected on a basis of the imaged image;
to detect a skeleton coordinate point indicating a body part of the occupant on the imaged image on a basis of the imaged image;
to detect a convulsion of the occupant on a basis of skeleton coordinate point information regarding the skeleton coordinate point detected;
to acquire biometric information of the occupant;
to detect pulse-related information regarding a pulse of the occupant on a basis of the biometric information acquired;
to detect a pulse abnormality of the occupant on a basis of the pulse-related information of the occupant detected;
to determine whether or not the occupant has a physical condition abnormality on a basis of whether or not the process has detected the posture collapse of the occupant, whether or not the process has detected the convulsion of the occupant, and whether or not the process has detected the pulse abnormality of the occupant;
to acquire vibration information regarding vibration of the mobile object; and
to remove noise included in the skeleton coordinate point information of the occupant on a basis of the skeleton coordinate point information of the occupant and the vibration information, wherein
the process detects the convulsion of the occupant on a basis of the skeleton coordinate point information of the occupant after removing the noise.

16.-17. (canceled)

18. A physical condition abnormality determination device comprising:
a processor; and
a memory storing a program to perform, upon executed by the processor, a process:
to detect posture collapse of an occupant in a mobile object on a basis of a head position of the occupant in an imaged image obtained by imaging at least a face of the occupant, the head position being detected on a basis of the imaged image;
to detect a skeleton coordinate point indicating a body part of the occupant on the imaged image on a basis of the imaged image;
to detect a convulsion of the occupant on a basis of skeleton coordinate point information regarding the skeleton coordinate point detected;
to acquire biometric information of the occupant;
to detect pulse-related information regarding a pulse of the occupant on a basis of the biometric information acquired;
to detect a pulse abnormality of the occupant on a basis of the pulse-related information of the occupant detected; and
to determine whether or not the occupant has a physical condition abnormality on a basis of whether or not the process has detected the posture collapse of the occupant, whether or not the process has detected the convulsion of the occupant, and whether or not the process has detected the pulse abnormality of the occupant, wherein
the process detects, in addition to the skeleton coordinate point of the occupant, the skeleton coordinate point indicating a body part of a passenger other than the occupant on the imaged image,
to remove noise included in the skeleton coordinate point information of the occupant on a basis of the skeleton coordinate point information of the occupant and the skeleton coordinate point information of the passenger, and
the process detects the convulsion of the occupant on a basis of the skeleton coordinate point information of the occupant after removing the noise.

19. A physical condition abnormality determination method comprising:
detecting posture collapse of an occupant in a mobile object on a basis of a head position of the occupant in an imaged image obtained by imaging at least a face of the occupant, the head position being detected on a basis of the imaged image;
detecting a skeleton coordinate point indicating a body part of the occupant on the imaged image on a basis of the imaged image;
detecting a convulsion of the occupant on a basis of skeleton coordinate point information regarding the skeleton coordinate point detected;
determining that the occupant has a physical condition abnormality when the posture collapse of the occupant is not detected and the convulsion of the occupant is detected;
acquiring vibration information regarding vibration of the mobile object; and
removing noise included in the skeleton coordinate point information of the occupant on a basis of the skeleton coordinate point information of the occupant and the vibration information, wherein
the method detects the convulsion of the occupant on a basis of the skeleton coordinate point information of the occupant after removing the noise.

20. A physical condition abnormality determination method comprising:
detecting posture collapse of an occupant in a mobile object on a basis of a head position of the occupant in an imaged image obtained by imaging at least a face of the occupant, the head position being detected on a basis of the imaged image;
detecting a skeleton coordinate point indicating a body part of the occupant on the imaged image on a basis of the imaged image;
detecting a convulsion of the occupant on a basis of skeleton coordinate point information regarding the skeleton coordinate point detected; and
determining that the occupant has a physical condition abnormality when the posture collapse of the occupant is not detected and the convulsion of the occupant is detected, wherein
the method detects, in addition to the skeleton coordinate point of the occupant, the skeleton coordinate point indicating a body part of a passenger other than the occupant on the imaged image, further comprising:
removing noise included in the skeleton coordinate point information of the occupant on a basis of the skeleton coordinate point information of the occupant and the skeleton coordinate point information of the passenger, and wherein
the method detects the convulsion of the occupant on a basis of the skeleton coordinate point information of the occupant after removing the noise.

21. A physical condition abnormality determination method comprising:
detecting posture collapse of an occupant in a mobile object on a basis of a head position of the occupant in an imaged image obtained by imaging at least a face of the occupant, the head position being detected on a basis of the imaged image;
detecting a skeleton coordinate point indicating a body part of the occupant on the imaged image on a basis of the imaged image;
detecting a convulsion of the occupant on a basis of skeleton coordinate point information regarding the skeleton coordinate point detected;
acquiring biometric information of the occupant;
detecting pulse-related information regarding a pulse of the occupant on a basis of the biometric information acquired;
detecting a pulse abnormality of the occupant on a basis of the pulse-related information of the occupant detected;
determining whether or not the occupant has a physical condition abnormality on a basis of whether or not the method has detected the posture collapse of the occupant, whether or not the method has detected the convulsion of the occupant, and whether or not the method has detected the pulse abnormality of the occupant;
acquiring vibration information regarding vibration of the mobile object; and
removing noise included in the skeleton coordinate point information of the occupant on a basis of the skeleton coordinate point information of the occupant and the vibration information, wherein
the method detects the convulsion of the occupant on a basis of the skeleton coordinate point information of the occupant after removing the noise.

22. A physical condition abnormality determination method comprising:
detecting posture collapse of an occupant in a mobile object on a basis of a head position of the occupant in an imaged image obtained by imaging at least a face of the occupant, the head position being detected on a basis of the imaged image;
detecting a skeleton coordinate point indicating a body part of the occupant on the imaged image on a basis of the imaged image;
detecting a convulsion of the occupant on a basis of skeleton coordinate point information regarding the skeleton coordinate point detected;
acquiring biometric information of the occupant;
detecting pulse-related information regarding a pulse of the occupant on a basis of the biometric information acquired;
detecting a pulse abnormality of the occupant on a basis of the pulse-related information of the occupant detected; and
determining whether or not the occupant has a physical condition abnormality on a basis of whether or not the method has detected the posture collapse of the occupant, whether or not the method has detected the convulsion of the occupant, and whether or not the method has detected the pulse abnormality of the occupant,
wherein
the method detects, in addition to the skeleton coordinate point of the occupant, the skeleton coordinate point indicating a body part of a passenger other than the occupant on the imaged image, further comprising:
removing noise included in the skeleton coordinate point information of the occupant on a basis of the skeleton coordinate point information of the occupant and the skeleton coordinate point information of the passenger, and wherein
the method detects the convulsion of the occupant on a basis of the skeleton coordinate point information of the occupant after removing the noise.
